(19) United States
(12) Patent Application Publication
(10) Pub. No.: US 2022/0243184 A1
(43) Pub. Date: Aug. 4, 2022

(54) ENGINEERED Cas-Transposon SYSTEM FOR PROGRAMMABLE AND SITE-DIRECTED DNA TRANSPOSITIONS

(71) Applicant: THE TRUSTEES OF COLUMBIA UNIVERSITY IN THE CITY OF NEW YORK, New York, NY (US)

(72) Inventors: Harris He Wang, New York, NY (US); Sway Chen, New York, NY (US)

(21) Appl. No.: 17/533,379

(22) Filed: Nov. 23, 2021

Related U.S. Application Data
(63) Continuation of application No. PCT/US2020/034538, filed on May 26, 2020.
(60) Provisional application No. 62/852,629, filed on May 24, 2019.
(60) Provisional application No. 62/946,201, filed on Dec. 10, 2019.
(60) Provisional application No. 62/963,938, filed on Jan. 21, 2020.

(51) Int. Cl. C12N 9/12; C12N 9/22; C12N 15/11; C12N 15/62; C12N 15/90

(52) U.S. Cl. CPC C12N 9/1241; C12N 9/22; C12N 15/11; C12N 15/62; C07K 2319/00; C12N 2310/20; C12N 2800/80; C12N 2800/10; C12N 2800/90; C12N 15/907

(57) ABSTRACT

Disclosed herein are systems, methods and components for targeted gene editing. Certain embodiments relate to a Cas protein lacking catalytic activity fused to a transposase. Also disclosed are systems that involve a Cas-transposase fusion protein, gRNA sequences and at least one mini-transposon for directing transpositions at user-defined genetic loci. Implementations of the system may involve disruption of a target gene or insertion of a payload sequence into a target nucleic acid.

### CROSS-REFERENCE TO RELATED APPLICATIONS

This application is a continuation of International Patent Application No. PCT/US2020/034538, filed May 26, 2020, which claims the benefit of U.S. Provisional Application Nos. 62/852,629 filed May 24, 2019, 62/946,201 filed Dec. 10, 2019, and 62/963,938 filed Jan. 21, 2020, the contents of each of which are herein incorporated by reference in its entirety.

### SEQUENCE LISTING STATEMENT

The text of the computer readable sequence listing filed herewith, titled “38842-302_SEQUENCE-LISTING_ST25”, created Apr. 19, 2022, having a file size of 105,972 bytes, is hereby incorporated by reference in its entirety.

### BACKGROUND

Genome engineering relies on molecular tools for targeted and specific modification of a genome to introduce insertions, deletions, and substitutions. While numerous advances have emerged over the last decade to enable programmable editing and deletion of bacterial and eukaryotic genomes, targeted genomic insertion remains an outstanding challenge.¹ Integration of desired heterologous DNA into the genome needs to be precise, programmable, and efficient—three key parameters of any genome integration methodology. Currently available genome integration tools are limited by one or more of these factors. Recombinases such as Flp² and Cre³ that mediate recombination at defined recognition sequences to integrate heterologous DNA have limited programmability.^{4,5} Site-specific nucleases such as CRISPR-associated (Cas) nucleases,^{6,7} zinc-finger nucleases (ZFNs),⁸ and transcription activator-like effector nucleases (TALENs)⁹ can be programmed to generate double-strand DNA breaks that are then repaired to incorporate a template DNA. However, this process relies on host homology-directed repair machinery, which is variable and often inefficient, especially as the size of the DNA insertion increases.¹⁰

Transposable elements are selfish genetic systems capable of integrating large pieces of DNA into both prokaryotic and eukaryotic genomes. Among various known transposable elements,^{11,12} the Himar1 transposon from the horn fly Haematobia irritans¹³ has been co-opted as a popular tool for insertional mutagenesis. The Himar1 transposon is mobilized by the Himar1 transposase, which like other Tel/mariner-family transposases, functions as a homodimer to bind the transposon DNA at the flanking inverted repeats, excise the transposon, and paste it into a random TA dinucleotide on a target DNA.¹³⁻¹⁶ Himar1 requires no host factors for transposition and functions in vitro,¹³ in bacteria,¹⁷ and in mammalian cells,¹⁸ and is capable of inserting transposons >7 kb in size.¹⁹ A hyperactive mutant of the transposase, Himar1C9, which contains two amino acid substitutions and increases transposition efficiency by 50-fold,²⁰ has enabled the generation of transposon insertion mutant libraries for genetic screens in diverse microbes.²¹⁻²³ However, because Himar1 transposons are inserted randomly into TA dinucleotides, their utility in targeted genome insertion applications has thus far been limited.

There has been great interest in harnessing the integration capabilities of transposases for genome editing. Synthetic approaches to increase the specificity of random transposon insertions aim to increase the affinity of the transposon or the transposase to specific DNA motifs. IS608, which is directed by base-pairing interactions between a transposon end and target DNA to insert 3′ to a tetranucleotide sequence, was shown to be targeted more specifically by increasing the length of the guide sequence in the transposon end.²⁴ However, altering transposon flanking end sequences affects the physical structure and biochemical activity of the transposon, limiting the range of viable sequence alterations that can be made. Several studies have described fusing transposases to DNA-binding protein (DBP) domains to direct transposon insertions to specific loci. Fusing the Gal4 DNA-binding protein to Mos1 (a Tc1/mariner family member) and piggyBac transposases increased the frequency of integration sites near Gal4 recognition sites.²⁵ Fusion of DNA-binding zinc-finger or transcription activator-like (TAL) effector proteins to piggyBac enabled integration into specified genomic loci in human cells.²⁶⁻²⁸ ISY100 transposase (also a Tc1/mariner family member) has been fused to a Zif268 Zinc-finger domain to increase specificity of transposon insertions to DNA adjacent to Zif268 binding sites.²⁹

More recently, researchers have begun uniting the powerful integration abilities of transposases with precision targeting by RNA-guided Cas nucleases to achieve targeted transposon integration. In nature, CRISPR-associated Tn7-like transposases have been discovered in cyanobacteria³⁰ and in Vibrio cholerae.³¹ In each of these studies, a Tn7-like transposase was found to be genetically encoded in close association with a CRISPR-Cas system. The RNA-guided Cas-effector complex was deficient in DNA cleavage but recruited the Tn7-like transposase protein subunits to insert transposons locally near its binding site, thereby enabling programmable insertions of transposons both in vitro and in vivo in Escherichia coli genomes. Other studies draw upon synthetic biology research showing that Cas nucleases can be repurposed as RNA-guided DNA-binding protein domains for manipulation of DNA sequences and gene expression at user-defined loci, in applications such as CRISPR interference (CRISPRi),^{32,33} CRISPR activation (CRISPRa),^{33,34} FokI-dCas9 dimeric nucleases,^{35,36} base editors,^{37,38} dCas9-targeted Gin serine recombinase,³⁹ and targeted histone modifiers.^{40,41} Likewise, transposases that naturally insert transposons randomly can be fused to catalytically dead Cas9 (dCas9) for targeted transposition. A recent study showed that a synthetic Himar1 transposase-dCas9 fusion protein enabled directed transposition in cell-free reactions.⁴²

### BRIEF DESCRIPTION OF THE DRAWINGS

FIG. 1A through FIG. 1E. Schematics of the in vitro Cas-Transposon (CasTn) test system. (FIG. 1A) Overview of Himar1-dCas9 protein function. The Himar1-dCas9 fusion protein is guided to the target insertion site by a gRNA, where it is tethered by the dCas9 domain. The Himar1 domain dimerizes with that of another fusion protein to cut-and-paste a Himar1 transposon into the target gene, which is knocked out in the same step. (FIG. 1B) Implementation of the CasTn system in vitro. Transposon donor and target plasmids were mixed with purified protein and gRNA. Following purification of transposition reactions, a mix of donor, target, and transposition product plasmids was obtained and analyzed by several assays. cmR, chloramphenicol resistance; GFP, green fluorescent protein; carbR, carbenicillin resistance; oriR, origin of replication. (FIG. 1C) Sodium dodecyl sulfate polyacrylamide gel electrophoresis of purified Himar-dCas9 protein. (FIG. 1D) Schematic of target plasmid-transposon junction polymerase chain reaction (PCR) assay. The PCR was performed using primer 1, which binds the transposon, and primer 2, which binds the target plasmid. Site-specific transposition results in an enrichment for a PCR product corresponding with the expected transposition product. PCR amplicons for transposition reactions containing gRNA-guided transposases and random, unguided transposases were analyzed by next-generation sequencing. (FIG. 1E) Schematic of transformation assay. In vitro reaction products were transformed into electrocompetent Escherichia coli to isolate single transposition events from individual colonies containing a transposition product, and to calculate the efficiency of transposition (fraction of all target plasmids bearing a transposon conferring chloramphenicol resistance).

FIG. 2A through FIG. 2C. Himar-dCas9 specificity is dependent on gRNA spacing and target site. (FIG. 2A) Illustration of gRNA strand orientation and spacings to TA insertion site gRNA1 (SEQ ID NO: 53) and gRNA2 (SEQ ID No: 54) and target DNA (SEQ ID No: 55). (FIG. 2B) PCR analysis of transposon-target junctions from in vitro reactions containing 30 nM Himar-dCas9/gRNA complex, 2.27 nM transposon donor DNA, and 2.27 nM target DNA. Reactions (n=3) were run using gRNAs with spacings between 5 and 18 bp from the TA insertion site. Non-targeting gRNA (gRNA_5), no gRNA, and no transposase controls were also performed. Arrowheads indicate expected site-specific PCR products for each gRNA. Error bars indicate standard deviation. (FIG. 2C) Transposon sequencing results for reactions with no gRNAs (left, n=4) or with gRNA_4 (n=3), gRNA_8 (n=3), gRNA_12 (n=3), or gRNA_5 (n=3). The baseline random distribution of transposons along the recipient plasmid in each panel with a gRNA is shown in light gray. Inset of position 5999 shows SEQ ID NO: 56.

FIG. 3A through FIG. 3F. Himar-dCas9-mediated site-directed transposition is robust to changes in ribonucleoprotein complex and DNA concentration. Target plasmids were pGT-B1 and donor plasmids were pHimar6. (FIG. 3A) PCR analysis of transposition reactions (n=3) using varying levels of Himar-dCas9/gRNA_4 complexes. Reactions were performed for 3 h at 30° C. with 5 nM donor and recipient plasmid DNA. (FIG. 3B) Transformation assay to measure transposition rates in reactions using varying levels of Himar-dCas9/gRNA_4 complexes (n=5). Reactions were performed for 3 h at 30° C. with 5 nM of donor and recipient plasmid DNA. (FIG. 3C) PCR analysis of transposition reactions (n=3) using varying levels of donor plasmid DNA. Reactions were performed for 3 h at 30° C. with 5 nM of recipient plasmid DNA and 30 nM Himar-dCas9/gRNA_4 complex. (FIG. 3D) PCR analysis of transposition reactions (n=3) using varying levels of recipient plasmid DNA. Reactions were performed for 3 h at 30° C. with 0.5 nM of donor plasmid DNA and 30 nM Himar-dCas9/gRNA_4 complex. (FIG. 3E) PCR analysis of transposition reactions (n=3) performed for different lengths of time in the presence or absence of background nonspecific DNA. Reactions were performed at 37° C. with 1 nM recipient plasmid DNA, 1 nM donor plasmid DNA, and 100 nM Himar-dCas9/gRNA_4 complex. Background E. coli genomic DNA was present at 10× the mass of recipient plasmid DNA. (FIG. 3F) Quantitative PCR measurement of transposition efficiency in reactions shown in panel (FIG. 3E). n=3 for each reaction condition. In all panels, arrowheads indicate the expected targeted transposition PCR product for gRNA_4, and error bars indicate standard deviation. Cq measurements correspond to log-scale differences in transposase activity.

FIG. 4A through FIG. 4E. Himar-dCas9 performs site-directed transposition into plasmids in E. coli. (FIG. 4A) Three plasmids were transformed into S17 E. coli to create a testbed for Himar-dCas9 transposition specificity in vivo. Post-transposition plasmids were extracted from the bacteria and analyzed by PCR and by transformation into competent E. coli with Sanger sequencing of plasmids from individual colonies. (FIG. 4B) To measure the ability of Himar-dCas9 to bind to a gRNA-specified target site in a bacterial cell, E. coli were transformed with the pTarget plasmid containing the green fluorescent protein (GFP) gene and an expression vector for Himar-dCas9 and one gRNA. Himar-dCas9 knocked down GFP expression in E. coli with gRNA_1, which targets the non-template strand (N) of the GFP gene. Himar-dCas9 did not knock down GFP fluorescence when expressed with a gRNA complementing the template strand (T) or with a non-targeting gRNA (NT) or no gRNA. These cells did not contain transposon donor DNA. n=2 per gRNA and ATC concentration; error bars indicate standard deviation. (FIG. 4C) PCR assay of in vitro transposition reactions using donor plasmid pHimar6 and recipient plasmid pTarget. Donor and recipient plasmids (2.27 nM each) along with 30 nM Himar-dCas9/gRNA complex were incubated for 3 h at 30° C. Expected PCR products of targeted insertions are shown with arrowheads. (FIG. 4D) PCR analysis of pTarget-transposon junctions resulting from in vivo transposition in bacteria. Three out of five gRNA_1 PCR products showed enrichment for the targeted insertion product. Transpositions A, B, C, and D with gRNA_1 were also analyzed by transformation and colony analysis. (FIG. 4E) Plasmid pools from four independent in vivo transposition experiments using gRNA_1 were transformed into E. coli, and the resultant colonies were analyzed by PCR and Sanger sequencing. The pie charts show the number of colonies containing on- and off-target transposition products from each plasmid pool, with the chart area proportional to the total number of colonies.

FIG. 5A through FIG. 5B. Himar1C9-dCas9 (Himar-dCas9) fusion protein retains DNA binding and transposition functionalities. (FIG. 5A) dCas9 and Himar-dCas9 were expressed in MG1655 galK::mCherry-specR E. coli with gRNAs 5 and 16. Protein expression was induced with aTc (0-100 ng/mL); n=3 for each condition. Both proteins decreased mCherry expression compared with the parent strain, indicating that the Himar-dCas9 fusion protein bound to the mCherry gene specified by the gRNAs and blocked transcription. (FIG. 5B) The transposition rates of Himar1C9 and Himar-dCas9 (without gRNA) were measured in an E. coli conjugation assay (n=3 for transposases, n=2 for control). Both Himar1C9 and Himar-dCas9 mediated transposition at higher rates than the no-transposase control. Error bars indicate standard deviation.

FIG. 6. Workflow for transposon sequencing library preparation from in vitro transposition reactions. To isolate transposons selectively that had become integrated into the target plasmid for sequencing, we performed PCRs using a biotinylated primer complementing the transposon end and reverse primers complementing the target plasmid. Two PCRs using reverse primers on opposite sides of the recipient plasmid were performed to account for PCR size bias during amplification of transposon junction products. PCR products were isolated using streptavidin beads and digested with MmeI to isolate transposon ends with a 17 bp overhang. A sequencing adapter was ligated, and the DNA was PCR amplified to add barcoded Illumina adapters. The resulting libraries from each PCR were sequenced independently and normalized for total reads, and the normalized libraries were averaged to obtain transposon insertion frequencies into each locus on the plasmid.

FIG. 7. gRNA-directed transposition is a property of Himar-dCas9 fusion proteins but not unfused Himar1C9 and dCas9. In vitro transposition reactions containing purified Himar-dCas9 with gRNA_4, Himar1C9 and dCas9 with gRNA_4, or no transposase were analyzed by a PCR assay for transposon-target plasmid junctions. Target plasmid was pGT-B1 (2.27 nM), and transposon donor was pHimar6 (2.27 nM). All protein concentrations were 30 nM.

FIG. 8. Quantitative measurement of Himar-dCas9 transposon insertions in the vicinity of gRNA target sites in cell-free in vitro reactions. These panels are zoomed-in graphs of transposon sequencing results from FIG. 2C for gRNA_4, gRNA_8, and gRNA_12, demonstrating that enrichment of gRNA-directed transposon insertions by Himar-dCas9 occurs at the TA nearest to the 5′ end of the gRNA. All TA sites are shown in red, while the protospacer adjacent motif (PAM) associated with each gRNA is bold underlined. Sequences shown are SEQ ID NOs: 14 and 57.

FIG. 9A through FIG. 9C. In vitro assay to analyze transposition by Himar-dCas9 with two gRNAs. (FIG. 9A) In vitro reactions containing two gRNAs were set up in two configurations to determine whether paired Himar-dCas9 proteins bound at the same TA site would improve transposase dimerization and activity compared to Himar-dCas9 proteins all bound individually to target plasmids. Himar-dCas9 was first incubated with either gRNA A (red) or gRNA B (blue), and then the Himar-dCas9-gRNA complexes were preloaded onto target plasmids as pairs (left) or as single complexes (right). Preloaded target plasmid-Himar-dCas9-gRNA complexes were then mixed with transposon donor plasmids. The total final concentration of each protein-gRNA complex was 2.5 nM, and final concentrations of donor and target DNAs were 5 nM. (FIG. 9B) PCR analysis of transposition by Himar-dCas9 with a single gRNA (left) or Himar-dCas9 with two gRNAs (right), preloaded in separated (S) or paired configurations (P). Arrowheads indicate PCR amplicons for site-specific transposon insertions for each reaction. (FIG. 9C) qPCR analysis of transposition by Himar-dCas9 with a single gRNA, Himar-dCas9 with two gRNAs (in a separated configuration), and Himar-dCas9 with two gRNAs (in a paired configuration). n=2-6 reactions per condition; error bars indicate standard deviation.

FIG. 10A through FIG. 10B. Transposon insertion in cell-free in vitro transposition reactions is not directionally biased. (FIG. 10A) Transposons can be inserted into a target locus in one of two orientations. For a given transposon insertion into the locus, directionality of the insertion can be determined by performing two PCRs, one amplifying each possible target-transposon junction, as only one PCR should produce a strong amplicon. (FIG. 10B) PCR screen of Stbl4 E. coli transformants of in vitro transposition products generated by Himar-dCas9 with gRNA_4 using 5 nM donor plasmid, 5 nM target plasmid, and 100 nM protein-gRNA complex. Out of 34 transformants with a transposon inserted into the GFP gene, there was a 19-15 split in the direction of transposon insertion.

FIG. 11A through FIG. 11C. Himar-dCas9 performs in vitro site-specific transposition in the presence of background DNA. (FIG. 11A) PCR analysis of transposition reactions (n=3-6) with varying levels of background E. coli genomic DNA. Reactions were performed for 3 h at 30 C with 1 nM target plasmid DNA, 1 nM donor plasmid DNA, and 10 nM Himar-dCas9-gRNA_4 complex. Ratios of background to target plasmid DNA were by mass. (FIG. 11B) PCR analysis of transposition reactions (n=3) performed for different lengths of time in the presence or absence of background nonspecific DNA. Reactions were performed at 37 C with 1 nM recipient plasmid DNA, 1 nM donor plasmid DNA, and 10 nM Himar-dCas9-gRNA_4 complex. Background E. coli genomic DNA was present at 10× the mass of recipient plasmid DNA. (FIG. 11C) qPCR measurement of transposition efficiency in reactions shown in panel (B). n=3 for each reaction condition. In all panels, error bars indicate standard deviation, and arrowheads indicate PCR amplicons for site-specific transposon insertions.

FIG. 12A through FIG. 12E. Himar-dCas9 was not observed to target transposon insertions into a genomic locus in CHO cells. (FIG. 12A) eGFP+ CHO cells were transfected with an expression vector for Himar-dCas9 and a mini-transposon donor vector with expression constructs for gRNAs targeting the eGFP gene. The mini-transposon contained a promoterless puromycin resistance gene and mCherry gene, which would both be expressed if the transposon integrated into the correct target site on eGFP. Puromycin-resistant cells resulting from transfection were analyzed by flow cytometry and PCR for transposon-target junctions. (FIG. 12B) PCR assay of in vitro transposition reactions with Himar-dCas9 and eGFP-targeting gRNAs, using donor plasmid pHimar6 and recipient plasmid pZE41-eGFP. Donor and recipient plasmids (2.27 nM) along with 30 nM Himar-dCas9-gRNA complex were incubated for 3 h at 37 C. Expected PCR products of targeted insertions are shown with arrowheads. gRNAs M1 and M2 target the same insertion site. (FIG. 12C) Representative flow cytometry dot plots for transfected cells after 13 days of puromycin selection. A transposase-free control transfection did not produce viable cells and was not analyzed by flow cytometry. (FIG. 12D) Upon flow cytometry, 5-15% of cells in some transfections were GFP−. (FIG. 12E) PCR for eGFP− transposon junctions in genomic DNA resulting from in vivo transposition did not show evidence of site-specific transposition. The positive control PCR used a plasmid with the transposon cloned into the target site of eGFP as template. The arrowhead indicates the expected size of the targeted transposition product, which is the same for gRNAs M1, M2, and M1+M2.

### DETAILED DESCRIPTION

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

The term “active fragment” as used herein with respect to amino acid sequences of polypeptides or proteins refers to a fragment of the referenced amino acid sequence, or defined variants thereof having a specified sequence identity, that exhibit the functional activity of the referenced amino acid sequence, or variants thereof. For example, an active fragment of a transposase enzyme encoded by SEQ ID NO:2 would be a fragment of this sequence that also exhibits transposase activity. An active fragment of a dCas9 protein would be a fragment that still associates with gRNA and binds to target DNA.

The terms “Cas” or “Cas protein”, as used herein their broadest sense, refer to a protein that associates with a gRNA and is guidable by the gRNA to a target nucleic acid. A “Cas enzyme” is a Cas protein that is able to cleave a target sequence (i.e. possesses nuclease activity). As is explained further herein, most embodiments utilize a Cas protein that has been mutated to lack catalytic activity (i.e. lack nuclease activity to cleave a target sequence).

As used herein, the term “Cas-transposase” refers to a fusion protein that comprises a Cas domain and a transposase domain. Typically, the Cas domain and transposase domain are fused via a linker.

The term “construct” or “gene construct” as used herein refers to a DNA sequence encoding a protein or RNA sequence that is associated with regulatory sequences which is inserted in the right orientation in a vector.

The term “effective amount,” as used herein, refers to an amount of a biologically active agent that is sufficient to elicit a desired biological response. For example, in some embodiments, an effective amount of a transposase may refer to the amount of the transposase that is sufficient to induce transposition at a target site specifically bound and recombined by the transposase. As will be appreciated by the skilled artisan, the effective amount of an agent, e.g., a nuclease, a transposase, a hybrid protein, a fusion protein, a protein dimer, a complex of a protein (or protein dimer) and a polynucleotide, or a polynucleotide, may vary depending on various factors as, for example, on the desired biological response, the specific allele, genome, target site, cell, or tissue being targeted, and the agent being used.

The term “engineered,” as used herein refers to a protein molecule, a nucleic acid, complex, substance, cell or entity that has been designed, produced, prepared, synthesized, and/or manufactured by a human. Accordingly, an engineered product is a product that does not occur in nature.

As used herein, the term “expression cassette” or “expression construct” refers to a unit cassette which includes a promoter and a polynucleotide encoding an expression product (polypeptide or RNA sequence), which is operably linked downstream of the promoter, to be capable of expressing the expression product. Various factors that can aid the efficient production of the expression product may be included inside or outside of the expression cassette. Conventionally, the expression cassette may include a promoter operably linked to the polynucleotide, a transcription termination signal, a ribosome-binding domain, and a translation termination signal. Specifically, the expression cassette may be in a form where the gene encoding the expression product is operably linked downstream of the promoter.

The term “fused” as used herein in reference to a protein refers to a connection of an end of a first protein domain with an end of second protein domain via a linker.

The term “guide RNA” or “gRNA” as used herein refers to an RNA molecule capable of directing a Cas enzyme to a target nucleic acid.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “linker,” as used herein, refers to a chemical group or a molecule linking two adjacent molecules or moieties, e.g., a binding domain (e.g., dCas9) and a transposase domain (e.g., Himar). In some embodiments, a linker joins a nuclear localization signal (NLS) domain to another protein (e.g., a Cas9 protein or a transposase or a fusion thereof). In some embodiments, a linker joins a gRNA binding domain of an RNA-programmable nuclease and the catalytic domain of a transposase. In some embodiments, a linker joins a dCas9 and a transposase. Typically, the linker is positioned between, or flanked by, two groups, molecules, or other moieties and connected to each one via a covalent bond, thus connecting the two. In some embodiments, the linker is an amino acid or a plurality of amino acids (peptide linker). In some embodiments, the linker is an organic molecule, group, polymer, or chemical moiety. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker comprises repeats of the tri-peptide Gly-Gly-Ser, e.g., comprising the sequence (GGS)ₙ, wherein n represents at least 1, 2, 3, 4, 5, 6, 7, 8, 9, 10, or more repeats. In some embodiments, the linker comprises the sequence (GGS)₆ (SEQ ID NO: 16). In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In another specific example, the linker implemented is an XTEN′ linker.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

“Nucleic acid” or “nucleic acid molecule” or “refers to deoxyribonucleotides or ribonucleotides and polymers thereof in either single- or double-stranded form. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Modifications of the ribose-phosphate backbone may be done to facilitate the addition of labels, or to increase the stability and half-life of such molecules in physiological environments. Nucleic acid analogs can find use in the methods of the invention as well as mixtures of naturally occurring nucleic acids and analogs. Furthermore, the polynucleotides herein may also be modified with a label capable of providing a detectable signal, either directly or indirectly. Exemplary labels include radioisotopes, fluorescent molecules, and biotin.

The term “optional” or “optionally” means that the subsequent described event, circumstance or substituent may or may not occur, and that the description includes instances where the event or circumstance occurs and instances where it does not.

The term “origin of replication,” as used herein, refers to a nucleic acid sequence in a replicating nucleic acid molecule (e.g., a plasmid or a chromosome) at which replication is initiated.

As used herein, “payload sequence” relates to any nucleic acid sequence encoding a payload. A payload sequence is typically, but not necessarily, heterologous to the cell into which they are introduced.

As used herein, the term “payload” refers to a peptide, polypeptide, protein, DNA and/or RNA sequence. Examples of payloads include, but are not limited to, therapeutic proteins, RNA interfering molecules, selectable markers (positive or negative e.g. auxotrophy, prototrophy or antibiotic resistance), reporter (e.g. fluorophore), and/or or nucleic acid sequences involved in genetic manipulation such as guide RNA sequences. Examples of reporter genes is found in Thorn, Mol Biol Cell, 2017, 28:848-857 incorporated herein. Examples antibiotic resistance markers include, but are not limited to, genes that confer resistance to ampicillin, carbenicillin, chloramphenicol, hygromycin B, kanamycin, spectinomycin, or tetracyline. At certain locations herein, the terms “payload” and “cargo” are used interchangeably. Examples of auxotrophic and prototrophic markers are described in U.S. Pat. No. 9,243,253, incorporated herein.

A “polynucleotide” or “nucleotide sequence” or “nucleic acid sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The term “polypeptide” or “amino acid sequence” as used herein means a compound of two or more amino acids linked by a peptide bond. “Polypeptide” is used herein interchangeably with the term “protein.”

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell and a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The term “RNA guide” as used herein refers to any RNA molecule that facilitates the targeting of a Cas protein described herein to a target nucleic acid. “RNA guides” include, but are not limited to, tracrRNAs, and crRNAs.

The term “sequence identity” or “identity,” as used herein in the context of two polynucleotides or polypeptides, refers to the residues in the sequences of the two molecules that are the same when aligned for maximum correspondence over a specified comparison window. As used herein, the term “percentage of sequence identity” or “% sequence identity” refers to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

The terms “target nucleic acid,” as used herein in the context of transposase, refers to a nucleic acid molecule that comprises at least one target site of a given transposase. In the context of fusions comprising a (nuclease-inactivated) RNA-programmable nuclease and a transposase domain, a “target nucleic acid” refers to one or more nucleic acid molecule(s) that comprises at least one target site. Non-limiting examples include target nucleic acids in a plasmid, in a genome or in a cell. In a more specific example, the target nucleic acid is in a prokaryote cell genome or eukaryote cell genome.

The term “target site” as used herein refers to the sequence of the target nucleic acid recognized by a given transposon for insertion. In some embodiments, the target nucleic acid(s) comprises at least two, at least three, or at least four target sites. In certain preferred embodiments, the target nucleic acid is in a bacterial genome.

The terms “trans-activating crRNA” or “tracrRNA” as used herein refer to an RNA including a sequence that forms a structure required for a Cas nuclease to bind to a specified target nucleic acid.

As used herein, the term “transposase” refers to an enzyme that binds to specific inverted repeat sequences flanking a transposon and catalyzes its movement from location to location in a polynucleotide or genome by a cut-and-paste mechanism or a replicative transposition mechanism. Examples of transposases include Himar1 and Tn5.

As used herein, the term “transposon” refers to a DNA sequence that can change its position (‘jump’) within a polynucleotide or genome. Transposons are flanked at both 5′ and 3′ ends by a specific inverted repeat DNA sequence that is recognized by the corresponding transposase protein. In a specific example, a transposon is a class II transposon whose movement from one location to another is governed by the activity of a cut-and-paste transposase.

The term “mini-transposon” or “MT” refers to an engineered transposon that does not contain a gene encoding a transposase protein. Mini-transposons are unable to self-mobilize and instead rely on exogenous transposase protein for mobilization, such as Cas-transposase described herein, in contrast with many naturally-occurring transposons that encode their own transposase and are self-mobilizing. MTs may be engineered to include a payload sequence, such that the payload sequence is inserted into a target site, and may be expressed to produce a payload. An MT may be inserted without a payload sequence, typically for the purpose of disrupting expression of the target nucleic acid.

As used herein, “transposon end sequence(s)” refer to sequences that are recognized by and bound by a specific transposase protein to initiate movement of a transposon. Transposon end sequences are typically short (˜15-30 bp) inverted repeat sequences flanking DNA transposons (including mini-transposons) on 5′ and 3′ ends. The 5′ inverted repeat sequence is the reverse complement of the 3′ inverted repeat. When the transposon “jumps,” the inverted repeats move with the transposon.

The terms “vector”, “cloning vector” and “expression vector” mean the vehicle by which a DNA or RNA sequence (e.g. a gene construct) can be introduced into a cell, so as to transform the cell and promote expression (e.g. transcription and translation) of the introduced sequence or knockdown or disruption of the target nucleic. Vectors include, but are not limited to, cells, plasmids, phages, and viruses.

Reference throughout this specification to “some embodiments”, “an embodiment,” “an example embodiment,” means that a particular feature, structure or characteristic described in connection with the embodiment is included in at least one embodiment of the present invention. Thus, appearances of the phrases “in some embodiments,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The recitation of numerical ranges by endpoints includes all numbers and fractions subsumed within the respective ranges, as well as the recited endpoints.

All publications, published patent documents, and patent applications cited herein are hereby incorporated by reference to the same extent as though each individual publication, published patent document, or patent application was specifically and individually indicated as being incorporated by reference.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### Overview

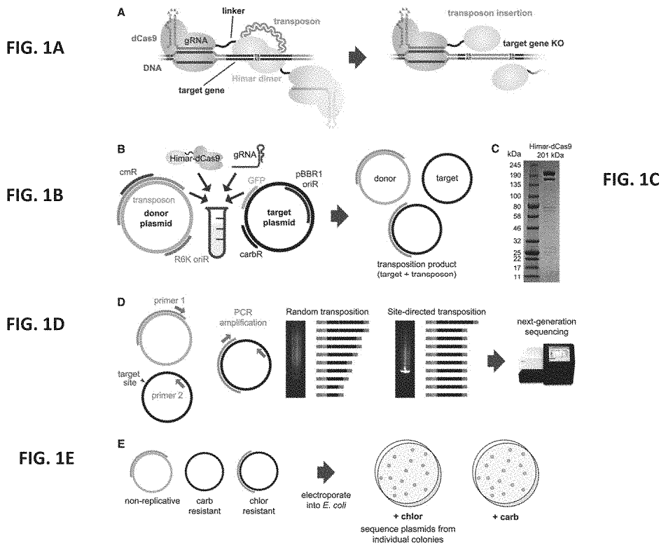

Disclosed herein is a novel technology, Cas-Transposon (CasTn), which unites the DNA integration capability of the Himar1 transposase and the programmable genome targeting capability of dCas9 to enable site-directed transpositions at user-defined genetic loci. This gRNA-targeted Himar1-dCas9 fusion protein integrates mini-transposons carrying synthetic DNA payload sequences of interest into specific loci with nucleotide precision (FIG. 1A), which has been demonstrated in both cell-free in vitro reactions and in a plasmid assay in E. coli. With further improvements to the system, CasTn can potentially function in a variety of organisms because the Himar1-dCas9 protein requires no host factors to function. An optimized CasTn platform may allow integration of a synthetic module of genes into a target locus, expanding the toolbox available to genome engineers in metabolic engineering⁴³ and emergent gene drive applications.⁴⁴

As set forth in the Examples, using cell-free in vitro assays, it has been demonstrated that the Himar-dCas9 fusion protein increased the frequency of transposon insertion at a single targeted TA dinucleotide by >300-fold compared to a random transposase, and that site-directed transposition is dependent on target choice while robust to log-fold variations in protein and DNA concentrations. It is also demonstrated that Himar-dCas9 mediates directed transposition into plasmids in Escherichia coli. This studies herein highlight CasTn as a new modality for host-independent, programmable, site-directed DNA insertions.

### Description of Exemplary Embodiments

### Cas-Transposase

Certain embodiments described herein pertain to a fusion protein comprising a transposase fused to a Cas protein (Cas-transposase). Typically, the fusion protein is capable of site-directed transposon insertions at user-defined genetic loci.

In a primary example, the Cas protein of the fusion protein is catalytically inactive, and the transposase is Himar1 or Tn5. In a specific example, the transposase comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO: 1 or active fragments thereof. In an alternative embodiment, the transposase comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO: 5 or active fragments thereof.

In a specific embodiment, the Cas nuclease of Cas-transposase is Cas9. In a more specific example, the Cas9 nuclease is catalytically dead. In further specific example, the Cas9 nuclease comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:3.

In an exemplary embodiment, the fusion protein is Himar1-dCas9. The Himar1-dCas9 may further comprise a linker between the transposase and the Cas nuclease. In a specific example, the linker comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:6.

### Cas

As is described a Cas protein is a protein that associates with a gRNA and is guidable by the gRNA to a target nucleic acid. The Cas protein may be able to cleave a target sequence (i.e. possess nuclease activity) or be mutated to lack catalytic activity (i.e. lack nuclease activity). Conventionally, the Cas enzyme directs cleavage of one or two strands at or near a target sequence, such as within the target sequence and/or within the complementary strand of the target sequence. For example, the Cas enzyme may direct cleavage of one or both strands within about 1, 2, 3, 4, 5, 6, 7, 8, 9, 10, 15, 20, 25, 50, 100, 200, 500, or more nucleotides from the first or last nucleotide of a target sequence. In certain embodiments, format on of a CRISPR complex results in cleavage (e.g., a cutting or nicking) of one or both strands in or near (e.g. within 1, 2, 3, 4, 5, 6, 7, 8, 9, 10, 20, 50, or more base pairs from) the target sequence. In some embodiments, the Cas enzyme lacks DNA strand cleavage activity.

The Cas enzyme may be a type II, type I, type III, type IV or type V CRISPR system enzyme. In some embodiments, the Cas enzyme is a Cas9 enzyme (also known as Csn1 and Csx12), preferably one mutated to lack catalytic activity. Non-limiting examples of the Cas9 enzyme include Cas9 derived from Streptococcus pyogenes (S. pyogenes), S. pneumoniae, Staphylococcus aureus, Neisseria meningitidis, Streptococcus thermophilus (S. thermophilus), or Treponema denticola. The Cas enzyme may also be derived from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

Non-limiting examples of the Cas enzymes also include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof.

Wildtype or mutant Cas enzyme may be used. In some embodiments, the nucleotide sequence encoding the Cas9 enzyme is modified to alter the activity of the protein. The mutant Cas enzyme may lack the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, D10A, H840A, N854A, N863A, and combinations thereof. In some embodiments, a Cas9 nickase may be used in combination with guide RNA(s), e.g., two guide RNAs, which target respectively sense and antisense strands of the DNA target.

Two or more catalytic domains of Cas9 (RuvC and/or HNH domains) may be mutated to produce a mutated Cas9 substantially lacking all DNA cleavage activity (a catalytically inactive Cas9). In some embodiments, a D10A mutation is combined with one or more of H840A, N854A, or N863A mutations to produce a Cas9 enzyme substantially lacking DNA cleavage activity (dead Cas 9 or dCas9). In some embodiments, a Cas enzyme is considered to substantially lack DNA cleavage activity when the DNA cleavage activity of the mutated enzyme is about or less than about 25%, 10%, 5%, 1%, 0.1%, 0.01%, or lower, compared to its non-mutated (wildtype) form. Other mutations may be useful; where the Cas9 or other Cas enzyme is from a species other than S. pyogenes, mutations in corresponding amino acids may be made to achieve similar effects.

The Cas protein can be introduced into a cell in the form of a DNA, mRNA or protein. The Cas protein may be engineered, chimeric, or isolated from an organism.

Another embodiment is a vector comprising one or more of the gRNA sequences and a nucleic acid sequence encoding a Cas-transposase. Alternatively, a sequence encoding a Cas-transposase may be provided in a vector separate from a vector encoding gRNA(s). In some embodiments, the vector comprises two or more Cas-transposase coding sequences operably linked to different promoters. In some embodiments, the host cell expresses one or more Cas-transposase(s) or gRNA(s).

### Gene Editing Systems and Methods

Other embodiments relate to systems to transpose a mini-transposon at a target site of a target nucleic acid. In one embodiment, the system includes a nucleic acid sequence that encodes a fusion protein comprising a Cas domain and transposase domain fused via a linker, such as the Cas-transposase described herein. The system further includes at least one gRNA sequence complementary to a segment of the target nucleic acid, wherein the segment is adjacent to a target site for mini-transposon insertion. In addition, the system may comprise at least one mini-transposon that is inserted at the target site in conjunction with the transposase used.

In embodiments where disruption of expression of a gene is desired, the mini-transposon implemented need not be fused with a payload sequence. All that would be required is that the mini-transposon be inserted at the target site, where the target site is one where the insertion disrupts expression (i.e. transcription or translation) of the target nucleic acid.

In other embodiments where the delivery of a payload, such as in a cell, is desired, a first transposon end sequence is fused to the 5′ end of payload sequence and a second transposon end sequence is fused to a 3′ end of a payload sequence.

In one implementation, the system may be configured for cell-free insertion of a mini-transposon at the target site. In this implementation, the components of the system may be naked sequences, or associated with a vector. Also, in an alternative embodiment, the system does not require expression of a sequence encoding the fusion protein. This would typically be in cell free utilization, wherein the actual fusion protein (e.g. Cas-transposase) is provided along with the gRNA. In this embodiment, the gRNA may be preloaded onto Cas-transposase before being provided to the target nucleic acid.

Where the target nucleic acid is within a cell, the components of the system are generally, though not necessarily, packaged in a vector, which can be in the form of a number of different configurations. For example, the system may include a first plasmid harboring a nucleic acid sequence encoding a Cas-transposase, a second plasmid harboring a gRNA nucleic acid sequence and a third plasmid harboring a mini-transposon (with or without a payload sequence). Alternatively, a combination at least two components of the system may be packaged in a vector, with any remaining components packaged in a separate vector. The arrangement can be in any number of different configurations so long as the required components for insertion of the mini-transposon are provided to the target nucleic acid. Specific versions are further described in the Examples section below.

The system may also be designed to insert a mini-transposon in a target nucleic acid in a cell in vivo. In such instance, a vector suitable for in vivo administration would be utilized, including but not limited to a virus such as retroviruses, adenoviruses, adeno-associated viruses, herpes simplex virus, and the like. See Lundstrom, Viral Vectors in Gene Therapy, Diseases, 2018, 6(2):42. Alternatively, components of the system are administered to a subject via naked polynucleotides (e.g. naked DNA), or physical vehicles such as liposomes and nanoparticles. It is noted that the above approaches for inserting a transposon in a cell in vivo, may be applied to cells in vitro. See Nayerossadat et al., Adv Biomed Res, 2012; 1:27.

In one example, the gRNA of the system typically comprises 15-25 bp. The gRNA sequence is optimally designed to have a segment that hybridizes to the target nucleic acid at a location 3-50 bp from the target site. In a more specific example, the gRNA includes a segment that hybridizes 5-30 bp from the target site.

Examples of mini-transposons that may be utilized in the system include, but are not limited to, gene constructs flanked by inverted repeat sequences of the Himar1 transposon and Tn5 transposon. Examples of specific Himar1 mini-transposons are found in the Sequences section herein below. However, permittable variations of the transposon end sequences can be implemented so long as they facilitate transposition at a target site. Accordingly, examples of transposon end sequences include sequences having at least 80%, at least 85%, at least 90%, at least 91%, at least 92%, at least 93%, at least 94%, at least 95%, at least 96%, at least 97%, at least 98% or at least 99% sequence identity with SEQ ID NO: 9 or SEQ ID NO:12.

Another embodiment pertains to a method of inserting a mini-transposon into a target site of a target nucleic sequence. The target nucleic acid may be in a cell-free system or in a cell. The method involves providing the target nucleic acid sequence with a fusion protein having a Cas domain and a transposase domain (e.g. Cas-transposase), at least one gRNA sequence complementary to a segment of DNA sequence, wherein the segment is adjacent to a target site for transposon insertion, and, optionally, at least one mini-transposon, that may or may not be fused to a payload sequence. The method is conducted under conditions to allow for insertion of the mini-transposon into the target site. The Cas domain and transposase domains are optionally fused via a linker. As described above, the insertion of the transposon may be conducted in an in vitro cell free system, in vitro cell system, or in a cell in vivo.

In a related embodiment, a method of inserting a payload sequence into a target site of a target nucleic acid is disclosed. The method involves providing to the target nucleic acid (i) a fusion protein having a Cas domain and a transposase domain (e.g. Cas-transposase), (ii) at least one gRNA sequence complementary to a segment of a target nucleic acid, wherein the segment is adjacent to the target site to direct transposon insertion; and (iii) a payload sequence comprising a 5′ end and a 3′ end, wherein the payload sequence comprises a first transposon end sequence fused to the 5′ end and a second transposon end sequence fused to the 3′ end. The method is conducted under conditions to allow for insertion of the mini-transposon-payload construct into the target site.

The elements of the system or elements provided to the targeted nucleic acid in the method embodiments may be packaged in one or more vectors. For example, (i) the fusion protein (e.g. Cas-transposase), (ii) the at least one gRNA, and (iii) the at least one mini-transposon or mini-transposon-payload construct may be packaged into a single vector, such as a plasmid or viral vector. In an alternative embodiment, two of elements (i), (ii), and (iii) are packaged into a first vector and a third element is packaged into a second vector. In another alternative embodiment, each of elements (i), (ii), and (iii) are packaged into a first, second and third vector, respectively. In a specific embodiment, the target nucleic acid is a DNA sequence in a cell.

According to a further embodiment, disclosed is an expression cassette including a nucleic acid sequence comprising a first nucleic acid sequence encoding a transposase, a second nucleic acid sequence encoding a Cas nuclease, and a third nucleic acid sequence encoding a linker peptide positioned between the first sequence and second sequence. In a specific example, the transposase pertains to Himar1 transposase or a Tn5 transposase. The transposase may comprise a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO: 1 or 2, or active fragments thereof. According to another example, the transposase comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:4, or active fragments thereof. In a specific example, the Cas domain of the expression cassette is Cas9. As discussed above, the Cas domain typically will encode a catalytically dead Cas protein. In a specific embodiment, the Cas9 nuclease comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:6, or active fragments thereof.

In a specific example, the nucleic acid sequence encoding the linker comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:6.

In another example, a Cas-transposase with linker comprises a polypeptide sequence comprising at least 80%, at least 85%, at least 90%, at least 91%, at least 92%, at least 93%, at least 94%, at least 95%, at least 96%, at least 97%, at least 98% or at least 99% sequence identity to the amino acid sequence of SEQ ID NO:3, SEQ ID NO:5, SEQ ID NO: 7 or SEQ ID NO:8. In an alternate embodiment, SEQ ID NO:3 includes one or more of the following mutations: Y12A, Y12S, F31A, W119A, V120A, P121A, R122A, E123A, L124A, and any combination thereof. In another alternate embodiment, SEQ ID NO:5 includes one or more of the following mutations: M470_I476del, A471_I476del, S458A and any combination thereof.

In related embodiments, provided are system embodiments comprising an expression cassette as described herein and at least one gRNA sequence complementary to a segment of DNA sequence, wherein the segment is adjacent to a target site of a target nucleic acid. In a specific embodiment, the segment is 15-25 bp in length. Typically, segment is 3-50 bp from the target site, or more specifically, 5-30 bp from the target site. Similar to other system embodiments described herein, the system may further include at least one mini-transposon. Where payload delivery is desired, at least one mini-transposon is fused with a payload sequence. In a more specific embodiment, a first transposon end sequence is fused to the 5′ end of a payload sequence and a second transposon end sequence that is fused at the 3′ end of the payload sequence. The transposon end sequences may be inverted repeats of a himar1 transposon or Tn5 transposon. In a specific embodiment, the transposon end sequence includes a sequence having at least 80%, at least 85%, at least 90%, at least 91%, at least 92%, at least 93%, at least 94%, at least 95%, at least 96%, at least 97%, at least 98% or at least 99% sequence identity with SEQ ID NO: 9, or the reverse complement thereof, or SEQ ID NO:12, or the reverse complement thereof. Typically, on a single strand nucleic acid sequence, the transposon end sequence on the 5′ end will be SEQ ID NO:9 or SEQ ID NO:12, and the transposon end sequence on the 3′ end reverse complement of SEQ ID NO:9 or SEQ ID NO:12, respectively.

Guide RNAs can be configured to have suitable lengths and distinct nucleic acid sequences to direct binding of a Cas-transposase adjacent to a target site of a target nucleic acid. In a specific example, the gRNA is configured to have a segment complementary to a location 3-50 bp from the target site. In a more specific example, the segment is complementary to a location 3-50 bp from the target site. Typically, the gRNA segment is 15-25 bp in length.

The gRNA is configured to bind to the Cas-transposase, which can be effectuated at different stages of the method. For example, the Cas-transposase may be pre-bound with gRNA prior to provision to target nucleic acid, which would typically be in the situation of an in vitro system. Alternatively, the Cas-transposase and gRNA are provided separately such as through expression by an expression cassette in a host cell and assembled within to allow the Cas-transposase to be guided to the target nucleic acid. Any guide sequence can be used in a gRNA, depending on the target nucleic acid. Considerations relevant to developing a gRNA include specificity, stability, and functionality. Specificity refers to the ability of a particular gRNA:Cas-transposase complex to bind to and/or cleave a desired target sequence, whereas little or no binding and/or cleavage of polynucleotides different in sequence and/or location from the desired target occurs. Thus, specificity refers to minimizing off-target effects of the gRNA:Cas-transposase complex. Stability refers to the ability of the gRNA to resist degradation by enzymes, such as nucleases, and other substances that exist in intracellular and extra-cellular environments. Further considerations relevant to developing a gRNA include transferability and immunostimulatory properties. Thus, gRNA are used that have efficient and titratable transferability into cells, especially into the nuclei of eukaryotic cells, and having minimal or no immunostimulatory properties in the transfected cells. Another important consideration for gRNA is to provide an effective means for delivering it into and maintaining it in the intended cell, tissue, bodily fluid or organism for a duration sufficient to allow the desired gRNA functionality.

As described in the Examples, the system and methods may implement more than one gRNA. For example, a first gRNA is configured to have a portion complementary to a segment of target nucleic acid sequence adjacent to a target site and a second gRNA configured to a have portion complementary to a segment of a target nucleic acid sequence adjacent to a target site. The first gRNA may bind to a segment on one strand of a double stranded DNA molecule, and the second gRNA may bind to a segment on the opposing strand of a double stranded DNA molecule.

Vectors may comprise a nucleic acid sequence into which a foreign nucleic acid sequence is inserted. A common way to insert one segment of nucleic acid sequence into another segment of a nucleic acid sequence involves the use of enzymes called restriction enzymes that cleave DNA at specific sites (specific groups of nucleotides) called restriction sites. A common type of vector is a “plasmid”, which generally is a self-contained molecule of double-stranded DNA, usually of bacterial origin, that can readily accept additional (foreign) DNA and which can readily introduced into a suitable cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors which replicate or exist episomally, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

Typically, an expression cassette is engineered such that it can be inserted into a vector at defined restriction sites. The cassette restriction sites are designed to ensure insertion of the cassette in the proper reading frame. Generally, a foreign nucleic acid is inserted at one or more restriction sites of the vector sequence, and then is carried by the vector into a host cell along with the transmissible vector sequence.

In other embodiments, provided is a kit comprising a container and any number of system elements described above. For example, the kit may comprise a Cas-transposase, at least one gRNA and/or at least one mini-transposon or mini-transposon/payload sequence construct, disposed either individually or in some combination in a container. In some applications, one or more system elements may be provided in pre-measured single use amounts in individual, typically disposable, tubes or equivalent containers. The kits can also include packaging materials for holding the container or combination of containers. Typical packaging materials for such kits and systems include solid matrices (e.g., glass, plastic, paper, foil, micro-particles and the like) that hold the system elements in any of a variety of configurations (e.g., in a vial, microtiter plate well, microarray, and the like). The kits may further include instructions recorded in a tangible form for use of the components.

In further embodiments, CasTn technology is implemented in vitro for purposes of exome capture, in which specific exons of interest from a genome are sequenced using high-throughput sequencing platforms. Historically, selected exons were captured for sequencing via hybridization with DNA probes (Albert T J, Molla M N, Muzny D M et al. Direct selection of human genomic loci by microarray hybridization. Nature methods. 2007; 4:903-905. DOI: 10.1038/nmeth1111; Parla J S, Iossifov I, Grabill I et al. A comparative analysis of exome capture. Genome biology. 2011; 12:R97. DOI: 10.1186/gb-2011-12-9-r97). CasTn offers an alternative mechanism for generating exome capture sequencing libraries. A purified fusion Cas-transposase, a library of guide RNAs (gRNAs) targeting exons of interest, and mini-transposons containing sequencing adapter sequences could be mixed in vitro with genomic DNA to enable selective insertion of sequencing adapters at the targeted exons. Exons flanked by adapters can then be amplified into a sequencing library by PCR. The reagents for this protocol (fusion transposase, mini-transposons, gRNA library, and PCR primers) may be made commercially available as a kit. Users would also be able to easily customize their exome capture by using custom-designed gRNAs and/or gRNA libraries.

In other embodiments, utilizations for in vivo CasTn technology include metabolic engineering. By delivering the components of CasTn, including a fusion Cas-transposase protein, one or more gRNAs targeting an endogenous gene, and a mini-transposon, into a cell, one could actuate the deletion of the targeted endogenous gene. Furthermore, by including a new gene or gene cassette on the mini-transposon, one could perform a one-step substitution of one gene for another, enabling facile manipulation of metabolic synthesis pathways. There are several possible embodiments for such a technology. The Cas-transposase could be delivered into a cell as a purified protein (via electroporation or liposome transfection), or encoded on a non-replicative plasmid to maintain stability of inserted transposons. gRNAs could be delivered either as purified gRNAs, either separately or associated with the Cas-transposase protein, or encoded on an expression vector such as a non-replicative plasmid. The transposon would be delivered on a nucleic acid vector such as a plasmid.

Summary of Results
a) A Cas-transposase comprising a catalytically inactive Cas9 domain fused with a Himar1 transposase was successfully produced.
b) An in vitro reporter system was devised involving a chlor resistance gene to test the ability of the Cas-transposase to successfully transposition transposons a site-directed loci. Studies using the reporter system demonstrated that the Cas-transposase successfully inserted the transposon chor resistance gene at intended loci on a GFP gene present on a recipient plasmid with high efficiency.
c) Studies demonstrated that the efficiency and site-specifity of transposon insertions was gRNA dependent.
d) The Cas-transposase fusion demonstrated robust transposition across a range of protein and DNA concentrations in vitro.
e) Cas-transposase was demonstrated to mediate site-directed insertions into plasmids in vivo in E. coli.

### EXAMPLES

### Example 1: Methods and Materials

### Strains, Media, and Growth Conditions

All E. coli strains were grown aerobically in LB Lennox broth at 37° C. with shaking, with antibiotics added at the following concentrations: carbenicillin (carb) 50 μg/mL, kanamycin (kan) 50 μg/mL, chloramphenicol (chlor) 20-34 μg/mL, and spectinomycin (spec) 240 μg/mL for S17 derivative strains and 60 μg/mL for non-S17 derivative strains. Supplements were added at the following concentrations: diaminopimelic acid (DAP) 50 μM, anhydrotetracycline (aTc) 1-100 ng/mL, and magnesium chloride (MgCl₂) 20 mM.

### Buffer Compositions

Buffers used in the study were as follows. Protein resuspension buffer (PRB): 20 mM Tris-HCl pH 8.0, 10 mM imidazole, 300 mM NaCl, 10% v/v glycerol. One tablet of cOmplete™ Mini, EDTA-free Protease Inhibitor Cocktail (Roche) was dissolved in 10 mL buffer immediately before use. Protein wash buffer (PWB): 20 mM Tris-HCl pH 8.0, 30 mM imidazole, 500 mM NaCl, 10% v/v glycerol. Protein elution buffer (PEB): 20 mM Tris-HCl pH 8.0, 500 mM imidazole, 500 mM NaCl, 10% v/v glycerol. Dialysis buffer 1 (DB1): 25 mM Tris-HCl pH 7.6, 200 mM KCl, 10 mM MgCl₂, 2 mM DTT, 10% v/v glycerol. Dialysis buffer 2 (DB2): 25 mM Tris-HCl pH 7.6, 200 mM KCl, 10 mM MgCl₂, 0.5 mM DTT, 10% v/v glycerol. 10× Annealing buffer: 100 mM Tris-HCl pH 8.0, 1 M NaCl, 10 mM EDTA (pH 8.1).

### Design and Construction of the Himar-dCas9 Transposase

The gene encoding fusion protein Himar1C9-XTEN-dCas9 (Himar-dCas9) was constructed from the hyperactive Himar1C9 transposase gene on plasmid pSAM-BT²¹ and the dCas9 gene from pdCas9-bacteria (Addgene plasmid #44249). Flexible peptide linker sequence XTEN³⁵ was synthesized as a gBlock® (Integrated DNA Technologies). DNA sequences were polymerase chain reaction (PCR) amplified using Kapa Hifi Master Mix (Kapa Biosystems) and cloned into expression vectors using NEBuilder® HiFi DNA Assembly Master Mix (New England Biolabs). Himar-dCas9 and Himar1C9 genes were cloned into a C-terminal 6×His-tagged T7 expression vector (yielding plasmids pET-Himar-dCas9 and pET-Himar) for protein production and purification. Himar-dCas9, dCas9, and Himar1C9 genes were cloned into tet-inducible bacterial expression vectors (yielding plasmids pHdCas9, pdCas9-carb, and pHimar1C9, respectively) to assess protein function in vivo. Tet-inducible bacterial expression vectors for Himar-dCas9 that additionally feature constitutive gRNA expression cassettes were constructed to evaluate site-specificity of Himar-dCas9 in vivo: pHdCas9-gRNA1, pHdCas9-gRNA4, pHdCas9-gRNA5, pHdCas9-gRNA5-gRNA16 containing gRNA_1, gRNA_4, gRNA_5, and both gRNA_5 and gRNA_16, respectively. Himar-dCas9 was cloned into a mammalian expression vector with an N-terminal 3×FLAG tag and SV40 nuclear localization signal (pHdCas9-mammalian), and this mammalian variant of the Himar-dCas9 protein was purified from C-terminal 6×His-tagged expression vector pET-Himar-dCas9-mammalian. Plasmids used in this study are described in Table 1. All gRNAs used in this study are described in Table 2.

Measurement of Himar-dCas9 Gene Expression Knockdown in E. coli

Expression knockdown of mCherry in E. coli strain EcSC83 (MG1655 galK::mCherry-specR) was measured. Tet-inducible expression vectors pHdCas9-gRNA5-gRNA16 and pdCas9-gRNA5-gRNA16 were used to produce either Himar-dCas9 or dCas9 (a positive control) in each strain along with two gRNAs targeting mCherry. Expression knockdown of green fluorescent protein (GFP) encoded on the pTarget plasmid in the E. coli S17 strain was measured. Tet-inducible expression vectors (pHdCas9-gRNA1, pHdCas9-gRNA4, pHdCas9-gRNA5, pHdCas9 for negative control) were used to express Himar-dCas9 along with a GFP-targeting gRNA in S17 with pTarget.

Saturated overnight E. coli cultures were diluted 1:40 into fresh LB media containing aTc to induce Himar-dCas9 or dCas9 expression. Aliquots of induced cultures (200 μL) were grown with shaking on 96-well plates at 37° C. on a BioTek plate reader. Measurements of OD600 and mCherry (excitation 580 nm, emission 610 nm) and GFP (excitation 485 nm, emission 528 nm) fluorescence were taken 12 h post induction.

Measurement of Himar-dCas9 Transposase Activity in E. coli

Himar-dCas9 and Himar1C9 proteins were expressed in MG1655 E. coli from tet-inducible expression vectors pHdCas9 and pHimar1C9, respectively. These strains were conjugated with DAP-auxotrophic donor strain EcGT2 (S17 asd::mCherry-specR)⁴⁵ containing transposon donor plasmid pHimar6, which has a 1.4 kb Himar1 mini-transposon containing a chlor resistance cassette and the R6K origin of replication, which does not replicate in MG1655.

Donor and recipient cultures were grown overnight at 37° C.; donors were grown in LB with DAP and kan, and recipients were grown in LB with carb. Donor culture (100 μL) was diluted in 4 mL fresh media. Recipient culture (100 μL) was diluted in 4 mL fresh media with 1 ng/mL aTc to induce transposase expression. Both cultures were grown for 5 h at 37° C. Donor and recipient cultures were centrifuged and re-suspended twice in phosphate-buffered saline (PBS) to wash the cells. Donor (10⁹) and recipient (10⁹) cells were mixed, pelleted, re-suspended in 20 μL PBS, and dropped onto LB agar with 1 ng/mL aTc. The cell droplets were dried at room temperature and then incubated for 2 h at 37° C. After conjugation, cells were scraped off, re-suspended in PBS, and plated±chlor (20 μg/mL) to select for recipient cells with an integrated transposon. Transposition rates were measured as the ratio of chlor-resistant colony-forming units (CFUs) to total CFUs.

### Purification of Himar-dCas9 Protein

His-tagged Himar-dCas9 was purified by nickel affinity chromatography from Rosetta2 cells (Novagen) bearing plasmid pET-Himar-dCas9 or pET-Himar-dCas9-mammalian. Saturated overnight culture (1 mL) grown in LB with chlor (34m/mL) and carb was diluted in 100 mL fresh media and grown to OD0.6-0.8 at 37° C. with shaking. Isopropyl β-d-1-thiogalactopyranoside (IPTG; 0.2 mM) was added to induce protein expression, and the flask was incubated for 16 h at 18° C. with shaking. The cells were pelleted by centrifugation at 7,197 g for 5 min at 4° C. and then re-suspended in 5 mL ice-cold PRB. Cells were lysed in an ice water bath using a Qsonica sonicator at 40% power for a total of 120 s in 20 s on/off intervals. The cell suspension was mixed by pipetting, and the sonication step was repeated. The lysate was centrifuged at 7,197 g for 10 min at 4° C. to pellet cell debris, and the cleared cell lysate was collected.

All subsequent steps were performed at 4° C. Ni-NTA agarose (1 mL; Qiagen) was added to a 15 mL polypropylene gravity flow column (Qiagen) and equilibrated with 5 mL of PRB. Cleared cell lysate was added to the column and incubated on a rotating platform for 30 min. The lysate was flowed through, and the nickel resin was washed with 50 mL PWB. The protein was eluted with PEB in five fractions of 0.5 mL each. Each elution fraction was analyzed by running an sodium dodecyl sulfate polyacrylamide gel electrophoresis. Elution fractions 2-4 were combined and dialyzed overnight in 500 mL DB1 using 10K MWCO Slide-A-Lyzer™ Dialysis Cassettes (Thermo Fisher Scientific). The protein was dialyzed again in 500 mL DB2 for 6 h. The dialyzed protein was quantified with the Qubit Protein Assay Kit (Thermo Fisher Scientific) and divided into single-use aliquots that were snap frozen in dry ice and ethanol and stored at −80° C. SDS-PAGE of purified Himar-dCas9 is shown in FIG. 1C.

### Purification of Himar1C9 Protein

C-terminal 6×His-tagged Himar1C9 was purified by nickel affinity chromatography from Rosetta2 cells (Novagen) bearing plasmid pET-Himar. Saturated overnight culture (1 mL) grown in LB with chlor (34m/mL) and carb was diluted in 100 mL fresh media and grown to OD0.9 at 37° C. with shaking. IPTG (0.5 mM) was added to induce protein expression, and the flask was incubated at 37° C. with shaking for 1 h. The cells were pelleted as described above, and the protein was purified using the His-Spin Protein Miniprep Kit (Zymo Research) according to the manufacturer's instructions, using the denaturing buffer protocol. The purified protein was dialyzed, frozen, and stored as described above. Purified Himar1C9 was used in control in vitro reactions along with commercially available purified dCas9 (Alt-R® S.p. dCas9 Protein V3; Integrated DNA Technologies).

### In Vitro Transposition Reaction Setup

The specificity and efficiency of transposition by purified Himar-dCas9 within in vitro reactions was characterized (FIG. 1B). Each reaction was performed in a buffer consisting of 10% glycerol, 2 mM dithiothreitol (DTT), 250 μg/mL bovine serum albumin (BSA), 25 mM HEPES (pH 7.9), 100 mM NaCl, and 10 mM MgCl₂. Plasmid DNA was purified using the ZymoPureII midiprep kit (Zymo Research). Background E. coli genomic DNA was purified using the MasterPure Gram Positive DNA Purification Kit (Epicentre). All DNAs were purified again using the Zymo Clean and Concentrator-25 Kit (Zymo Research) to remove all traces of RNAse. gRNAs were synthesized using the GeneArt™ Precision gRNA Synthesis Kit (Invitrogen). Concentrations of DNAs and gRNAs were measured using a Qubit 4 fluorometer (Invitrogen).

To set up in vitro reactions, frozen aliquots of Himar-dCas9 protein and gRNAs were thawed on ice. The protein was diluted to a 20× final concentration in DB2 buffer, and gRNAs were diluted to the same molarity in nuclease-free water. The diluted protein and gRNA were mixed in equal volumes and incubated at room temperature for 15 min. Transposon donor DNA, target plasmid DNA, and background DNA (if applicable) were mixed on ice with 10 μL 2× transposition buffer master mix and water to reach a volume of 18 μL. The protein/gRNA mixture (2 μL) was added last to the reaction. In reactions where the transposase/gRNA complex was preloaded onto the target plasmid, the target plasmid was mixed with protein and gRNA and incubated at 30° C. for 10 min, and donor DNA was added last. Transposition reactions were incubated for 3-72 h at 30-37° C. and then heat inactivated at 75° C. for 20 min. Transposition products were purified using magnetic beads⁴⁶ and eluted in 45 μL nuclease-free water.

### Quantitative PCR Assay for Site-Specific Insertions in Transposition Reactions

One method used to evaluate the specificity and efficiency of Himar-dCas9 within in vitro transposition reactions was a series of quantitative PCRs (qPCRs; FIG. 1D). For each reaction, two qPCRs were performed to obtain the measure of relative Cq: one PCR amplifying transposon-target plasmid junctions, and another PCR amplifying the target plasmid backbone to normalize for template DNA input across samples. Relative Cq values shown in this study are the differences between the two Cq values.

For in vitro transposition into pGT-B1 (target plasmid used in in vitro experiments), primers p433 and p415 were used for junction PCRs, and primers p828 and p829 were used for control PCRs. For in vitro transposition into pTarget (target plasmid used for in vivo bacteria experiments) or pZE41-eGFP (target plasmid used to test mammalian CasTn components in vitro), primers p898 and p415 were used for junction PCRs, and primers p899 and p900 were used for control PCRs. All qPCR primers used in this study are listed in Table 3.

### Transposon Sequencing Library Preparation

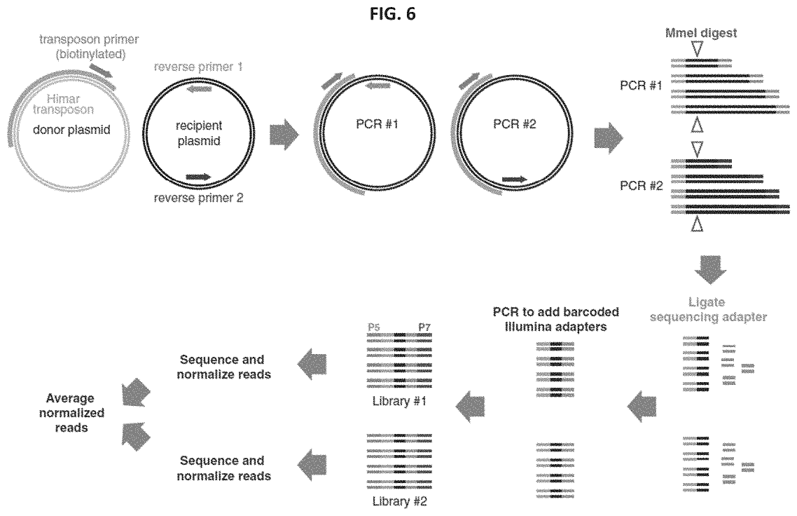

To survey the distribution of transposition events performed by Himar-dCas9, transposon sequencing was performed on in vitro reaction products (FIG. 6). Transposon junctions were PCR amplified from transposition reactions using primer sets p923/p433 and p923/p922 with Q5 HiFi 2× Master Mix (NEB)+SYBR Green. Primer p923 binds the Himar1 transposon from pHimar6, while p433 and p922 bind to target plasmid pGT-B1. PCR reactions were performed on a Bio-Rad C1000 touch qPCR machine with the same thermocycling conditions described in the qPCR protocol, but were stopped in the exponential phase to avoid oversaturation of PCR products. PCR products were purified using magnetic beads,⁴⁶ and 100-200 ng DNA per sample was digested with MmeI (NEB) for 1 h in a reaction volume of 40 μL. The digestion products were purified using Dynabeads M-270 streptavidin beads (Thermo Fisher Scientific) according to the manufacturer's instructions. The digested transposon ends, bound to magnetic Dynabeads, were mixed with 1 μg sequencing adapter DNA (see next section), 1 μL T4 DNA ligase, and T4 DNA ligase buffer in a total reaction volume of 50 μL. The ligations were incubated at room temperature (˜23° C.) for 1 h, and then the beads were washed according to the manufacturer's instructions and re-suspended in 40 μL water.

Dynabeads (2 μL) were used as a template for the final PCR using barcoded P5 and P7 primers and Q5 HiFi 2× Master Mix (NEB)+SYBR Green. Reactions were thermocycled using a Bio-Rad C1000 touch qPCR machine for 1 min at 98° C., followed by cycles of 98° C. denaturation for 10 s, 67° C. annealing for 15 s, and 72° C. extension for 20 s until the exponential phase. Equal amounts of DNA from all PCR reactions were combined into one sequencing library, which was purified and size selected for 145 bp products using the Select-a-Size Clean and Concentrator Kit (Zymo). The library was quantified with the Qubit dsDNA HS Assay Kit (Invitrogen) and combined at a ratio of 7:3 with PhiX sequencing control DNA. The library was sequenced using a MiSeq V2 50 Cycle Kit (Illumina) with custom read 1 and index 1 primers spiked into the standard read 1 and index 1 wells. Reads were mapped to the pGT-B1 plasmid using Bowtie 2.⁴⁷

### Construction of Sequencing Adapter

Oligonucleotides Adapter_T and Adapter_B were diluted to 100 μM in nuclease-free water. Ten microliters of each oligo was mixed with 2.5 μL water and 2.5 μL 10× annealing buffer. The mixture was heated to 95° C. and cooled at 0.1° C./s to 4° C. to yield 25 μL of 40 μM sequencing adapter, which was stored at −20° C.

### Transformation Assay for In Vitro Transposition Reaction Products

Another method used to measure transposition specificity and efficiency was transformation of the reaction product DNA into competent E. coli and analyzing transposon inserts in individual transformants (FIG. 1E). Purified DNA (5 μL) from an in vitro transposition reaction was mixed with 45 μL distilled water and chilled on ice. Thawed MegaX electrocompetent E. coli (10 μL; Invitrogen) was added and mixed by pipetting gently. The mixture was transferred to an ice-cold 0.1 cm gap electroporation cuvette (Bio-Rad) and electroporated at 1.8 kV. Cells were recovered in 1 mL SOC and incubated with shaking at 37° C. for 90 min. The cells were plated on LB+chlor (34m/mL) to select for target plasmids (pGT-B1) containing transposons, and on LB+carb to measure the electroporation efficiency of pGT-B 1. The efficiency of transposition was measured as the ratio of chlor-resistant transformants to carb-resistant transformants. To assess specificity of inserted transposons, we performed colony PCR on transformants using the primer set p433/p415 with KAPA2G Robust HotStart ReadyMix (Kapa Biosystems) to amplify junctions between the Himar1 transposon from pHimar6 and the pGT-B1 target plasmid, which were analyzed by Sanger sequencing. Although this primer set was expected to amplify only the junctions arising from transposon insertions in a single orientation (not the reverse orientation), due to recombination and inversion of the transposon in some MegaX cells after transformation, this PCR was sensitive enough to detect the location of the transposon insertion into pGT-B1 in all colonies, but not the direction of the transposon.

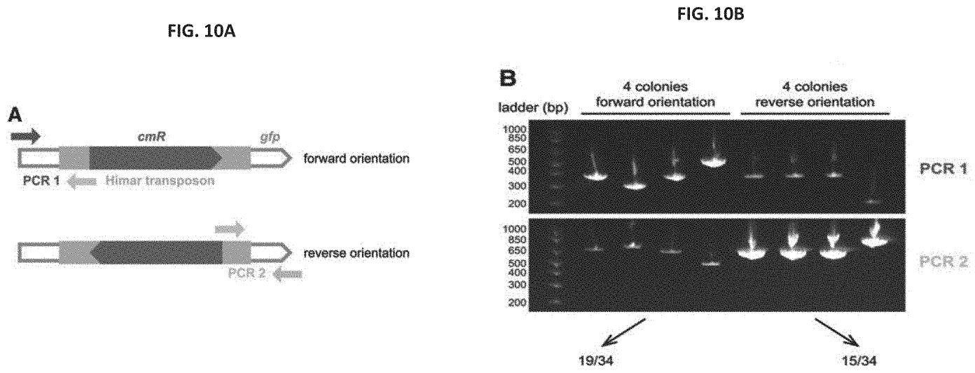

To assess the direction of transposon insertion into pGT-B1 plasmids, ElectroMAX™ Stbl4™ electrocompetent E. coli, which have lower rates of recombination, were transformed with DNA from in vitro transposition reactions as described above. We performed colony PCR on transformants using primer sets p771/p415 (amplifying “forward” transposon-target junctions) and p433/p415 (amplifying “reverse” junctions) to assess for directionality (FIG. 10).

In Vivo Assays for Transposition into a Target Plasmid

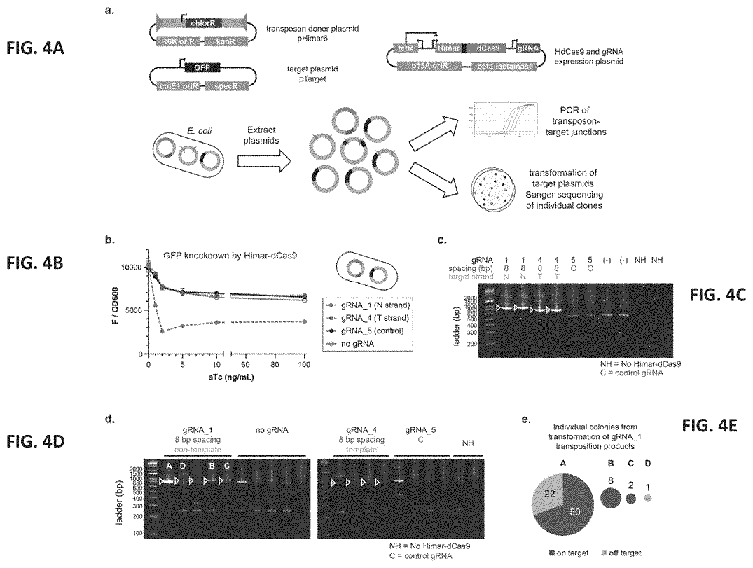

S17 E. coli were sequentially electroporated with plasmid pTarget as a target plasmid and then one of several pHdCas9-gRNA plasmids (pHdCas9-gRNA1, pHdCas9-gRNA4, pHdCas9-gRNA5, or pHdCas9), which are bacterial expression vectors for Himar-dCas9 and a gRNA (FIG. 4A and Table 1). Transformants were selected on LB with carb and spec (240 μg/mL). Transformants were grown from a single colony to mid-log phase in liquid selective media, electroporated with 130 ng pHimar6 transposon donor plasmid DNA, and recovered in 1 mL LB for 1 h at 37° C. with shaking post electroporation. One hundred microliters of a 10⁻³ dilution of the transformation was plated on LB agar plates with spec (240 μg/mL), carb, chlor (20 μg/mL), MgCl₂ (20 mM), and aTc (0-2 ng/mL). Plates were grown at 37° C. for 16 h. Between 10³ and 10⁴ colonies were scraped off each plate into 2 mL PBS and homogenized by pipetting. The cells (500 μL) were miniprepped using the QIAprep kit (Qiagen).

Minipreps from each transformation were evaluated by qPCR for junctions between the transposon from pHimar6 and the pTarget plasmid and by a transformation assay. qPCR assays for transposon-target plasmid junctions were performed as described above, using primers p898 and p415 and 10 ng miniprep DNA as PCR template. The control PCR to normalize for pTarget DNA input was performed with primers p899 and p900. In transformations, 150 ng plasmid DNA was electroporated into 10 μL MegaX electrocompetent cells diluted in 50 μL ice-cold distilled water. Cells were immediately recovered in 1 mL LB and incubated with shaking at 37° C. for 90 min. The cells were plated on LB agar with chlor (20 μg/mL) and spec (60 μg/mL) to select for pTarget plasmids containing a transposon from pHimar6. Colony PCR was performed using the primer set p898/p415 with KAPA2G Robust HotStart ReadyMix (Kapa Biosystems) to amplify transposon-pTarget junctions, which were analyzed by Sanger sequencing.

### Generation of Chinese Hamster Ovary Cell Lines for Transposition Assays

Chinese hamster ovary (CHO) cells were cultured in Ham's F-12K (Kaighn's) Medium (Thermo Fisher Scientific) with 10% fetal bovine serum and 1% penicillin-streptomycin. The eGFP+ CHO cell line was generated by transfection of plasmids pcDNA5/FRT/Hyg-eGFP and pOG44 into the Flp-In™-CHO cell line (Thermo Fisher Scientific) followed by selection in media with hygromycin (500m/mL). An eGFP−, mCherry+, puromycin-resistant site-specific transposition positive control cell line was generated by transfection of plasmids pcDNA5/FRT/Hyg-Himar and pOG44 into the Flp-In™-CHO cell line followed by selection in media with puromycin (10 μg/mL). Transfections were performed on cells at 70% confluence on six-well plates using 12 μL of Lipofectamine 2000 and 1,000 ng of each plasmid. Antibiotic selection was initiated 48 h after transfection. Polyclonal transfected cells were trypsinized and passaged for use in subsequent experiments.

### In Vivo Transposition Assays in Mammalian Cells

The eGFP+ CHO cell line was transfected with a pHP plasmid (transposon donor and gRNA expression vector) and the pHdCas9-mammalian expression plasmid. Transfections were performed on cells at 70% confluence on six-well plates using 12 μL of Lipofectamine 2000 and 1,250 ng of each plasmid. In the transposition negative control, the pHP-M1-M2 plasmid was transfected without the pHdCas9-mammalian plasmid. Transfection efficiencies were 40-70% based on flow cytometry measurements of mCherry expression in cells 24 h post transfection of control plasmid pHP-on. Antibiotic selection with puromycin (10m/mL) was initiated 48 h after transfection. Cells from each transfection were trypsinized after 9 days of selection, and the whole volume was transferred into a single well of a 12-well plate and grown for four more days in puromycin media. During 13 days of antibiotic selection, the medium was changed every 24 h. Post-selection cells were trypsinized and diluted 1:5 in fresh media and analyzed on a Guava easyCyte flow cytometer (Millipore). Gates for mCherry and GFP fluorescence were set using mCherry−/eGFP− CHO cells, mCherry−/eGFP+ CHO cells, and mCherry+/eGFP− transposition positive control CHO cells.

Genomic DNA from trypsinized cells was extracted using the Wizard Genomic DNA Purification Kit (Promega) for PCR analysis. qPCR for transposon-gDNA junctions was performed as described above using primers p933 and p946. The control PCR to normalize for DNA input was performed using primers p931 and p932. Purified gDNA (10 ng per sample) was used as PCR template.

### Example 2: Design of an Engineered Programmable, Site-Directed Transposase Protein

The design of the CasTn system leverages key insights from previous studies on Himar1 transposases and dCas9 fusion variants.^{7,20,29,32,34-36} The dCas9 protein is a well-characterized catalytically inactive Cas9 nuclease from Streptococcus pyogenes that contains the D10A and H840A amino acid substitutions^{7,32} and has been used as an RNA-guided DNA-binding protein for transcriptional modulation.³²⁻³⁴ Himar1C9 is a hyperactive Himar1 transposase variant that efficiently catalyzes transposition in diverse species and in vitro,²⁰ highlighting its robust ability to integrate without host factors in a variety of cellular environments. The C-terminus of Himar1C9 was fused to the N-terminus of dCas9 using flexible protein linker XTEN³⁵ (N-SGSETPGTSESATPES-C, SEQ ID NO: 52), as previous studies have described fusing other proteins to the N-terminus of dCas9 and to the C-terminus of mariner-family transposases.^{29,35,36}

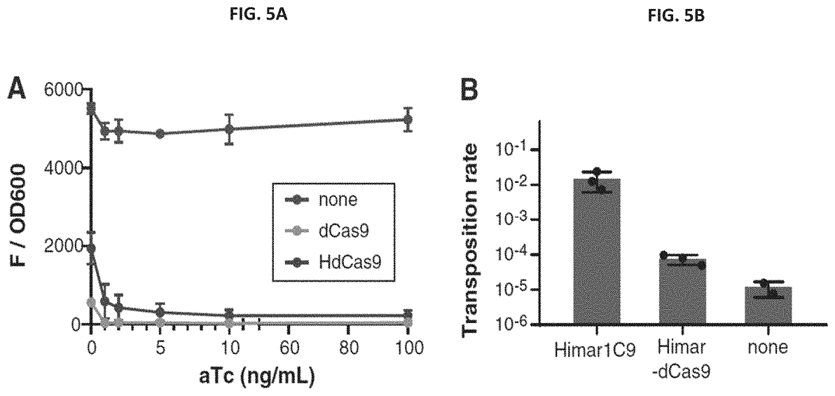

Because Himar1C9-dCas9 (Himar-dCas9) is a novel synthetic protein, it was verified that both the Himar1 and dCas9 components remained functional. To check that Himar-dCas9 was capable of binding a DNA target specified by a gRNA, Himar-dCas9 was expressed in an E. coli strain with a genomically integrated mCherry gene, along with two gRNAs targeting mCherry (gRNA_5 and gRNA_16 in Table 2). Knockdown of mCherry expression was observed, indicating that the DNA binding functionality of Himar-dCas9 was intact (FIG. 5A). To verify Himar-dCas9 transposition activity, a Himar1 mini-transposon was conjugated with a chloramphenicol resistance gene (on plasmid pHimar6) from EcGT2 donor E. coli into MG1655 E. coli expressing Himar-dCas9 or Himar1C9 transposase. The transposition rate was measured as the proportion of recipient cells that acquired a genomically integrated transposon (FIG. 5B). Himar-dCas9 mediates transposition events in E. coli, although at a lower rate (about 2 log-fold) compared with Himar1C9, which may be associated with lower expression of Himar-dCas9, which is a much larger and metabolically costly protein to produce, or with altered DNA affinity by dCas9, even in the absence of gRNA.⁴⁸

### Example 3: An In Vitro Reporter System to Assess Site-Directed Transpositions by Himar-dCas9

To establish and optimize parameters for site-directed transposition, an in vitro reporter system was developed to explore the transposition activity of Himar-dCas9. Purified Himar-dCas9 protein was mixed with transposon donor plasmid pHimar6 (containing a Himar1 mini-transposon with a chlor resistance gene), a transposon target pGT-B1 plasmid (containing a GFP gene), and one or more gRNAs targeted to various loci along GFP (FIG. 1B and Tables 1 and 2). Transposon insertion events into the pGT-B1 plasmid were analyzed by several assays. First, quantitative PCR (qPCR) of target plasmid-transposon junctions, using one primer designed to anneal to a part of the transposon DNA and one primer designed to anneal to a part of pGT-B1, enabled qualitative assessment of transposition specificity based on enrichment of qPCR products of the expected amplicon size, as well as quantitative estimation of transposition rate (FIG. 1D and Table 3). For every transposon-target junction qPCR, also performed was a control qPCR that amplifies the target plasmid's backbone to control for variations in DNA input between samples. Relative Cq measurements, an estimation of transposition efficiency, were taken as the difference between the Cq values from the junction and control qPCR reactions. Next-generation transposon sequencing (Tn-seq) further enabled measurement of the distribution of inserted transposons within the target plasmid (FIG. 1D and FIG. 6). Finally, transposition reaction products were transformed into competent E. coli to probe the specificity of transposition insertion sites further (FIG. 1E). Because the donor pHimar6 plasmid has a R6K origin of replication that is unable to replicate in E. coli without the pir replication gene, transformants containing the target pGT-B1 plasmid with an integrated transposon were. Transposition efficiency was determined by dividing the number of chloramphenicol-resistant transformants (CFUs with a target plasmid carrying a transposon) by the number of carbenicillin-resistant transformants (total CFUs with a target plasmid). Sanger sequencing of the target plasmid from chloramphenicol-resistant transformants revealed the site of integration and the transposition specificity.

### Example 4: Efficiency and Site-Specificity of Himar-dCas9 Transposon Insertions is gRNA Dependent

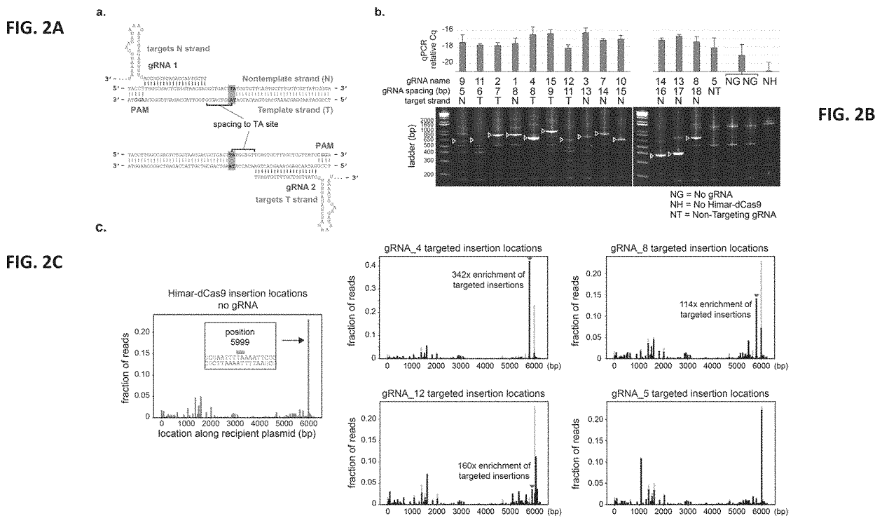

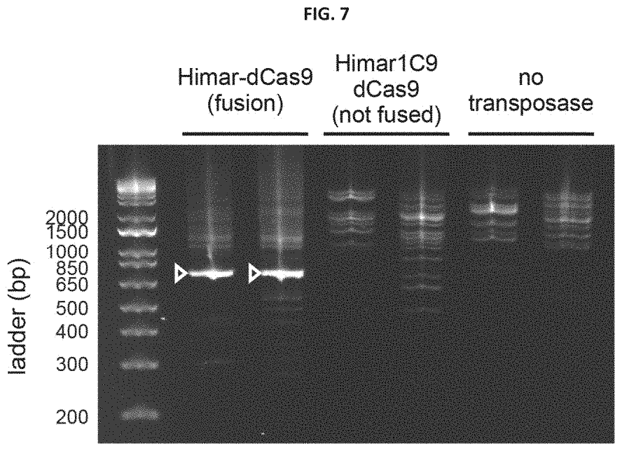

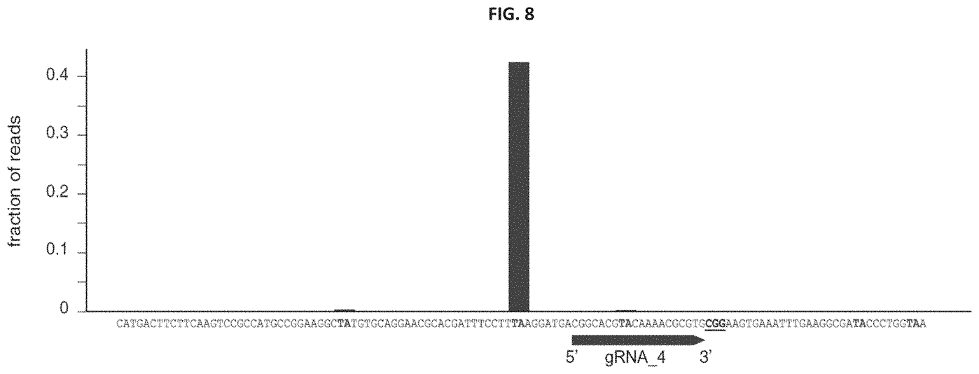

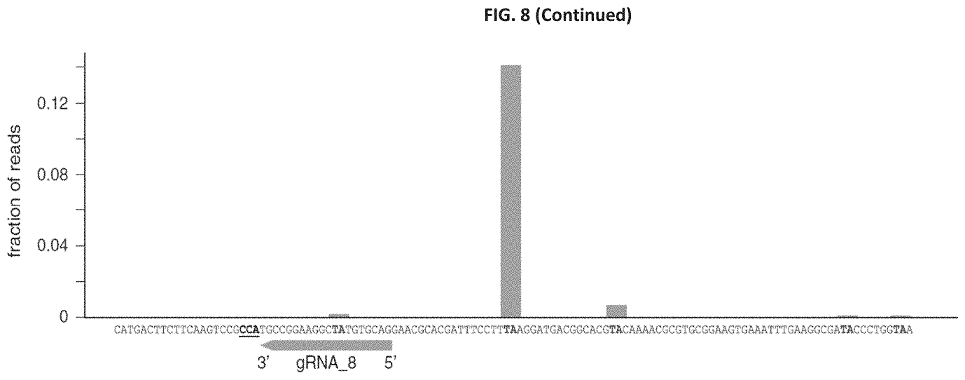

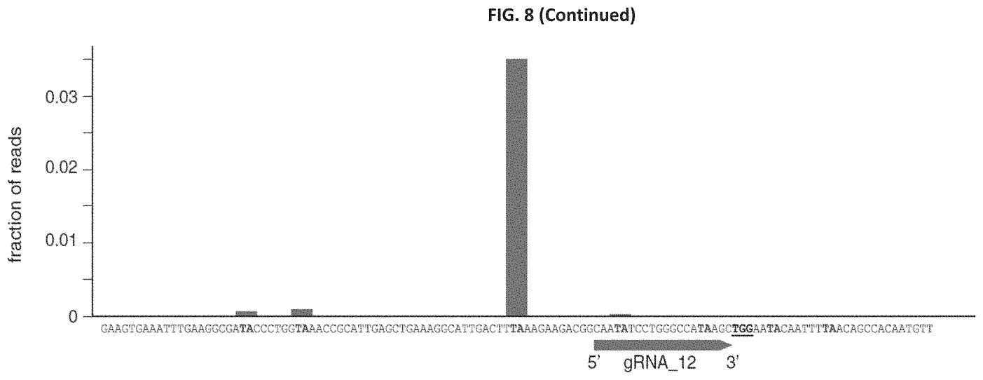

Using the in vitro reporter system, first assessed was how the orientation of the gRNA relative to the target TA dinucleotide affects the site specificity of transposition. gRNAs spaced 5-18 bp from a TA site, targeting either the template or non-template strand of GFP were tested (FIG. 2A and Table 2). Using the qPCR assay, it was found that a single gRNA is sufficient to effect site-directed transposition by Himar-dCas9, but not by unfused Himar1C9 and dCas9, indicating that Himar-dCas9 bound to a target site mediates transposition locally (FIG. 2B and FIG. 7). The site-specificity of these insertions is dependent on the gRNA spacing to the target TA site. All gRNA-directed insertion events occurred at the nearest TA distal to the 5′ end of the gRNA, as evidenced by gel purification and Sanger sequencing of enriched PCR bands (FIG. 2B) and by transposon sequencing of reaction products (FIG. 8). Site-directed transposition was robust in reactions using gRNAs with 7-9 bp and 16-18 bp spacings, but did not occur at all at short spacings (5-6 bp), likely due to steric hindrance by Himar-dCas9 at short distances. At spacings of 11-13 bp, there was a very faint expected PCR band, indicating that site-directed transposition at those sites was relatively poor. Slightly stronger bands at 14-15 bp spacings indicate intermediate performance of Himar-dCas9 in site-directed transposition. These findings are consistent with the previously observed spacing dependence for FokI-dCas9 proteins that use the same XTEN peptide linker.³⁵ The bimodal distribution of robustly targeting gRNA spacings may be due to the DNA double helix providing steric hindrance, since optimal spacings are approximately one helix turn (˜10 bp) apart.

To assess the distribution of transposon insertions around the target pGT-B1 plasmid, transposon sequencing was performed on transposition products resulting from three GFP-targeting gRNAs (gRNA_4, gRNA_8, and gRNA_12), a non-targeting gRNA, and no gRNA (FIG. 2C and FIG. 8). Although these distributions may not represent the true abundance of transposition events at each location, since sequencing was performed on size-biased PCR amplicons of transposon-target junctions, transposon distributions could be compared across reactions. The baseline distribution of random transposon insertions was generated from reactions with no gRNA. Random insertions were present throughout the 6.2 kb pGT-B1 plasmid, with a spike in transposition abundance at position 5999, a TA site in the middle of a 12 bp stretch of T/A nucleotides. This result is consistent with the observation that Himar1 transposase preferentially inserts transposons into flexible, T/A-rich DNA.⁴⁹ In contrast, gRNA-directed insertions were less likely to be inserted into position 5,999 and were enriched at their respective gRNA-adjacent TA sites compared with baseline (FIG. 2C). gRNA_4, with an optimal spacing of 8 bp from the target TA site, produced the best-targeted insertions, with 42% of sequenced transposon insertions being exactly at the target site, a 342-fold enrichment over baseline. Comparison of targeted insertion fold-enrichment across different gRNAs suggests that the specific target site and flanking DNA play a role in the specificity of transposon integration. For instance, gRNA_12 had a higher fold-enrichment of insertions at its target site than gRNA_8, but a lower fraction of measured insertions, suggesting that the target site of gRNA_12 may be intrinsically disfavored for transposition. Together, these results further show that Himar-dCas9 mediates directed transposon insertion to an intended integration site with the help of an optimally spaced gRNA.

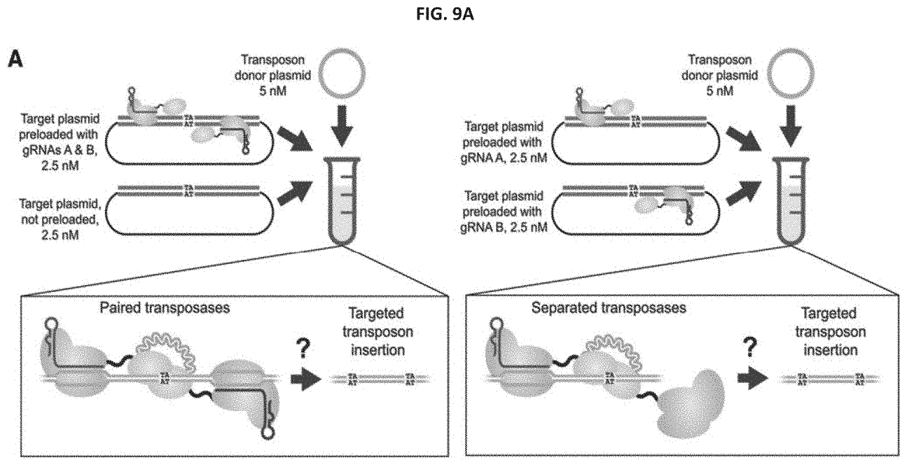

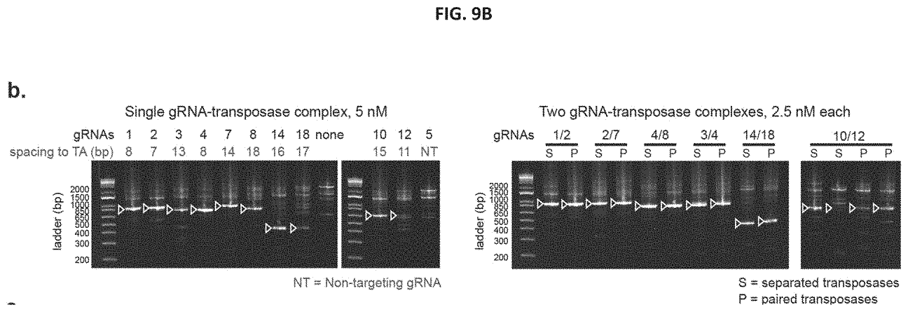

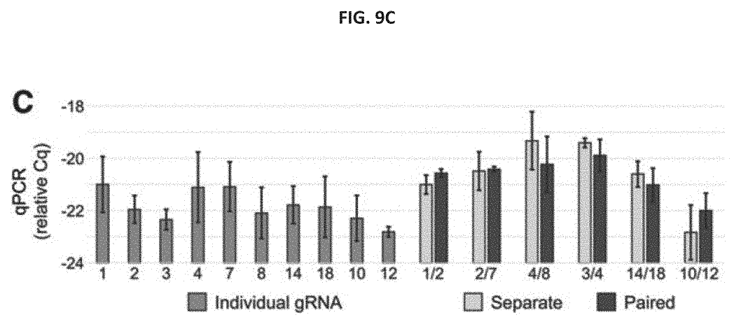

Given that mariner transposases dimerize in solution in the absence of DNA,⁵⁰ it was hypothesized that Himar-dCas9 dimerizes spontaneously, and the active Himar1 dimer is guided to a gRNA-specific target locus by one of the dCas9 domains in the Himar-dCas9 dimer (FIG. 1A). This mechanism is consistent with the observation that one gRNA is sufficient to direct targeted transposition. Further support for this hypothesis comes from in vitro reactions containing pairs of gRNAs targeting the same TA site but complementing opposite strands (FIG. 9). If Himar1 subunits did not spontaneously dimerize, then dimerization of Himar-dCas9 would be enhanced by loading two monomers onto the same target plasmid in close proximity. Reactions were devised in which target DNA was first preloaded with either paired or single gRNA/Himar-dCas9 complexes and then mixed with transposon donor DNA (FIG. 9A). In these experiments, the final reaction contained 5 nM Himar-dCas9, 5 nM donor DNA, 5 nM target DNA, and 2.5 nM each of two gRNAs. No difference in transposition rate or specificity between the gRNA/Himar-dCas9 complexes preloaded as pairs or as singletons was observed (FIG. 9B and FIG. 9C). The observation that preloading pairs of Himar-dCas9 complexes does not improve transposition is consistent with the hypothesis that transposase dimers formed before one of the gRNA/dCas9 domains targeted the dimer to its final location.

### Example 5: Site-Directed Transposition by Himar-dCas9 is Robust Across a Range of Protein and DNA Concentrations In Vitro

To assess the robustness of Himar-dCas9 to various experimental conditions and to determine the optimal parameters for site-directed transposition, different concentrations of (1) protein-gRNA complexes, (2) transposon donor plasmid (pHimar6) DNA, (3) target plasmid (pGT-B1) DNA, and (4) background off-target DNA within in vitro transposition reactions containing a single gRNA (gRNA_4) were explored. Also performed were in vitro reactions over different temperatures and reaction times.

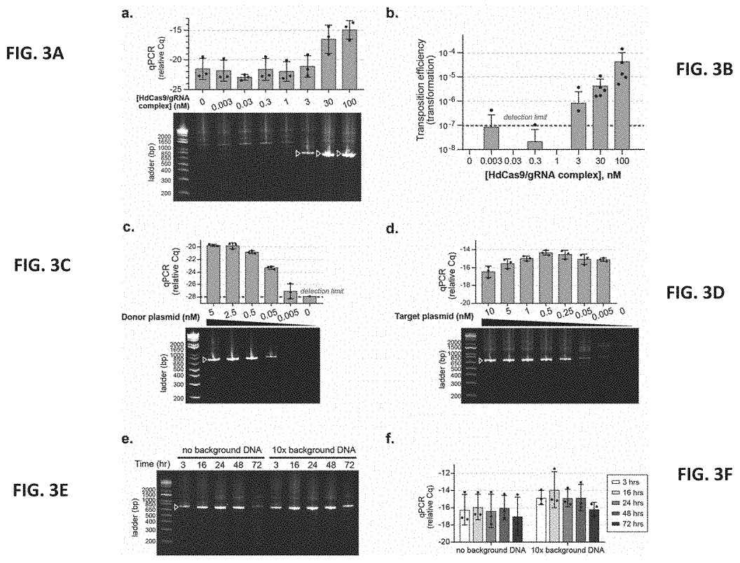

Varying concentrations of Himar-dCas9/gRNA complexes, site-directed transposition by PCR in in vitro reactions was detected with at least 3 nM of Himar-dCas9/gRNA complexes, using 5 nM donor and 5 nM target plasmids (FIG. 3A). Increasing the Himar-dCas9/gRNA concentration increased the yield of targeted transposition events. The trend of higher transposition rates at higher transposase concentrations was confirmed by the transformation assay (FIG. 3B), which also enabled precise analysis of transposition specificity from individual transformants. At 30 nM Himar-dCas9/gRNA complex, the specificity of transposon insertion into the targeted TA site was 44% (11/25 colonies). The specificity of insertion at 100 nM of the complex remained stable at 47.5% (19/40 colonies). The directionality of transposons inserted into the GFP gene was split approximately 50/50 based on screens of transformants (FIG. 10), supporting the hypothesis that insertion of transposons in a cell-free reaction is not directionally biased.

Next, it was explored whether site-directed transposition was affected by DNA concentrations of the donor or target plasmids. Using 5 nM target plasmid DNA, transposition activity was robust across 0.05-5 nM of donor plasmid DNA, with greater rates of transposition at higher donor DNA concentrations (FIG. 3C). Similarly, using 0.5 nM of donor plasmid DNA, site-directed transposition occurred across target plasmid concentrations of 0.25-10 nM (FIG. 3D). While the absolute rate of transposition (as assessed by Cq of the transposon-target junction qPCR) was higher at higher target DNA concentrations, the relative Cq remained relatively stable across target DNA concentrations, indicating that a similar proportion of target plasmids received a transposon in each reaction.

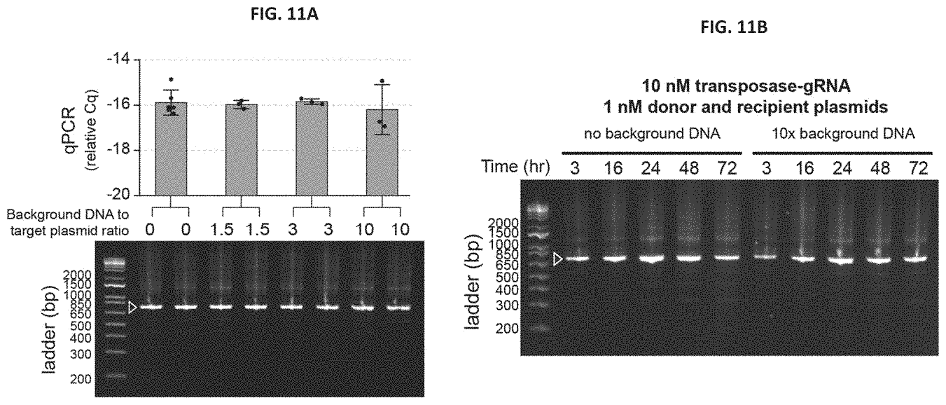

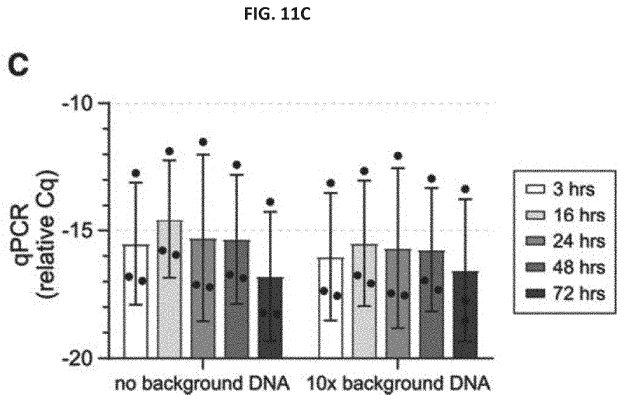

It was also tested whether the gRNA-guided Himar-dCas9 could efficiently transpose into a targeted site in the presence of background DNA and whether the amount of transposition changed over longer reaction times. Up to 10×(by mass) more background E. coli genomic DNA than target plasmid DNA to was added to in vitro transposition reactions. Across different ratios of target-to-background DNA concentrations tested, Himar-dCas9 was able to locate the gRNA-targeted site and insert transposons with no observed loss of specificity or efficiency (FIG. 11A). When similar reactions were performed containing 10× background DNA at 37° C. and over longer time courses instead of the standard protocol of 30° C. for 3 h, to mimic conditions in living cells, similar results were observed (FIG. 11B and FIG. 11C and FIG. 3E and F). The relative Cq and PCR band intensity of transposon-target junctions increased slightly between 3 and 16 h, suggesting that gRNA-guided transposases are faster at locating the target site than catalyzing transposition and that the increase in site-specific transposon insertions over time is performed by gRNA-dCas9 bound transposases. After 16 h, site-specific transposition events reached a plateau; the loss of specific transposon-target junctions observed at 72 h by PCR is likely due to degradation of reaction components (FIG. 11B and FIG. 3E).

Together, these results highlight that Himar-dCas9/gRNA mediates site-directed transposon insertions across a range of experimental conditions, including physiologically relevant temperatures and reactant concentrations. In bacteria, 1 nM corresponds to approximately one molecule per cell, while in eukaryotic cells, 1 nM corresponds to approximately 1,000 molecules per cell.⁵¹ Targeted transposition was observed to occur at protein concentrations of 1-100 nM (1-100 molecules of protein per bacterium) and DNA concentrations of <1 to 10 nM (1-10 DNA copies per bacterium). In bacteria, these concentrations are physiologically achievable with low protein expression and with transposon donor/target DNA present as a single chromosomal copy or on a low/medium copy number plasmid. Notably, no experimentally upper limit of protein/DNA concentrations was found for effective site-directed transposition beyond the loss of specific targeting due to increased background transpositions. Nevertheless, the CasTn system can be used with different plasmid expression systems to modulate copy numbers of both protein and DNA.

### Example 6: Himar-dCas9 Mediates Site-Directed Transposon Insertions into Plasmids In Vivo in E. coli

Since Himar-dCas9 robustly facilitated site-directed transposon integration in vitro, the ability of Himar-dCas9 to mediate site-specific transposition in two in vivo systems in E. coli and in mammalian cells was tested. In the first system, a set of three plasmids were transformed into S17 E. coli: pTarget, which contains a GFP target gene; pHimar6, the transposon donor plasmid; and a tet-inducible expression vector for Himar-dCas9 and a gRNA (FIG. 4A). These cells were grown on selective agar plates with MgCl₂ and anhydrotetracycline (aTc) to enable transposition and then extracted all plasmids. Transposition specificity was determined by two methods: PCR of transposon-target plasmid junctions, and transformation of plasmids into competent cells and analysis of transposon insertions in transformants.

It was first verified that the Himar-dCas9 system components functioned in vivo. By measuring transcriptional repression of GFP in E. coli containing pTarget and one of several Himar-dCas9/gRNA expression vectors, it was confirmed that gRNAs targeted Himar-dCas9 to the pTarget plasmid and determined the optimal concentration of aTc for inducing Himar-dCas9 expression (FIG. 4B). Consistent with previously reported results, gRNA_1, which targets the non-template strand of GFP, caused knockdown of GFP expression, but gRNA_4, which targets the template strand and does not sterically hinder RNA polymerase, did not cause GFP knockdown.³² Himar-dCas9 concentrations reached saturation at aTc induction levels of 2 ng/mL, as further increasing the concentration of aTc did not result in further knockdown of GFP by gRNA_1. It was also validated that purified Himar-dCas9 protein with gRNA_1 or gRNA_4 mediated targeted transposition into the GFP gene of pTarget in vitro (FIG. 4C).

In the in vivo assay, S17 E. coli containing pTarget, a Himar-dCas9/gRNA expression, and pHimar6 were grown on agar plates containing a saturating concentration of MgCl₂ and 1 ng/mL aTc to induce expression of Himar-dCas9 while avoiding overproduction inhibition of Himar1C9.⁵² After 16 h of growth at 37° C., we analyzed the pooled plasmids from all colonies for site-specific transposon insertions. PCR for transposon-target plasmid junctions showed that gRNA_1 produced detectable site-specific transposon insertions into pTarget in three out of five independent replicates (FIG. 4D). gRNA_4, however, did not produce an enrichment of PCR products corresponding to its target site.

The site specificity of transposition was further evaluated by transforming the plasmid pools into E. coli and analyzing individual transformants by colony PCR and Sanger sequencing in order to confirm that Himar-dCas9 with gRNA_1 mediated precisely targeted transposon insertions into pTarget. In three out of four independent replicates with gRNA_1, transformations produced colonies with mostly or all site-specific transposition products (FIG. 4E). In transformations of four plasmid pools from cells without a gRNA, no transformants were obtained with a transposon integrated into pTarget. Taken together, these results demonstrate in vivo directed transposition by an engineered Himar-dCas9 system for the first time.

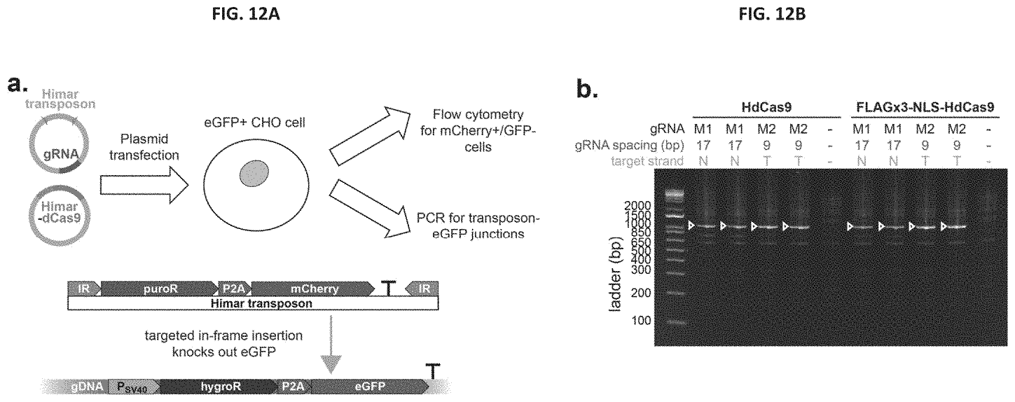

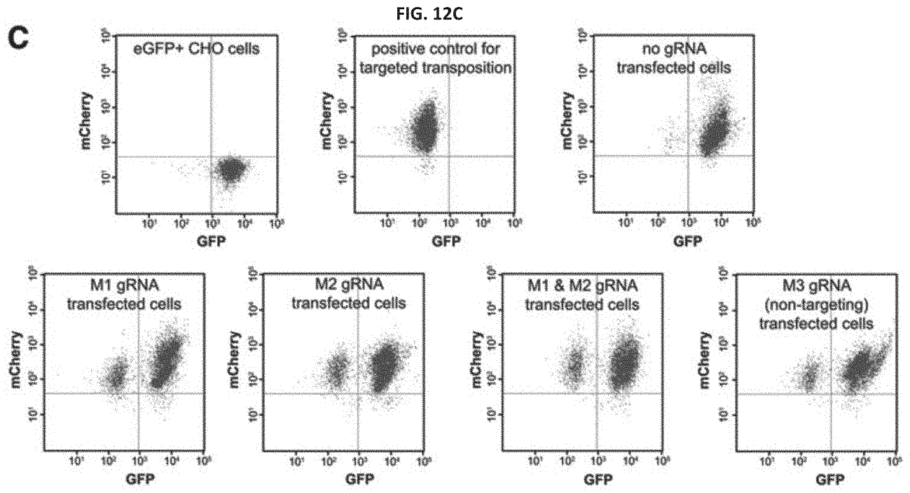

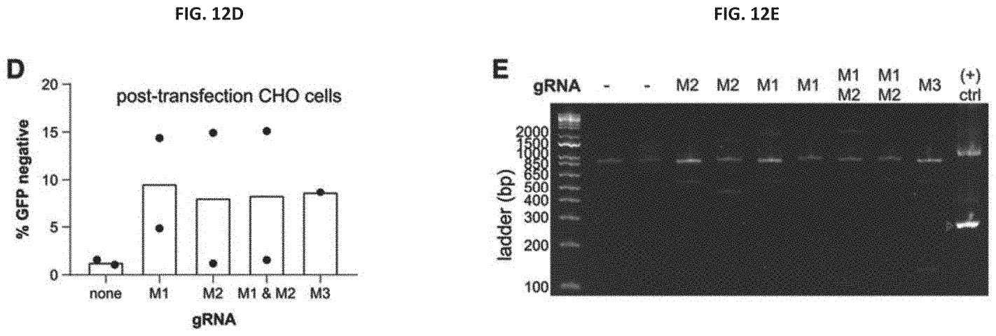

In a second in vivo test system, the ability of Himar-dCas9 to mediate site-specific transposition into a genomic locus in CHO cells was tested. CHO cells containing a single-copy constitutively expressed genomic eGFP gene were transfected with two plasmids: one containing a Himar transposon and gRNA expression operons, and the other being a Himar-dCas9 expression vector (FIG. 12A). The mammalian Himar-dCas9 was fused to an N-terminal 3×-FLAG tag and SV40 nuclear localization signal (NLS) and a C-terminal 6×-His tag. Two gRNAs were designed to target the eGFP gene at the same TA insertion site, complementing opposite strands. These gRNAs were tested individually and as a pair, along with a non-targeting gRNA and no gRNA. In vitro experiments demonstrated that the two gRNAs individually mediated site-specific transposition by the purified 3×-FLAG-NLS-Himar-dCas9-6×His protein (FIG. 12B).

The Himar transposon contained a promoterless puromycin resistance gene and mCherry gene, both of which would be inserted in-frame into the eGFP locus and expressed if targeted by Himar-dCas9 in the correct orientation (FIG. 12A). Because the transposon genes would only be expressed if the transposon were integrated downstream of a genomic promoter, puromycin selection for transposon mutants was stringent against false-positive clones resulting from plasmid integration into the genome. It was verified that transposon insertions into the target locus resulted in successful expression of puromycin resistance and mCherry by constructing a positive control cell line with the transposon cloned into that locus (FIG. 12C).

Following transfection, cells with an integrated transposon using puromycin were selected. From each transfection of approximately 10⁶ cells, About 20 colonies representing independent transposition events were obtained. Negative controls for transposition, which were transfected with only the transposon donor plasmid, did not produce viable cells, indicating clean selection against background plasmid integration events. All colonies from each transfection were pooled for analysis by flow cytometry and PCR for transposon-target junctions. Transfections with no gRNA resulted in few eGFP− cells, while some transfections with at least one gRNA (including the non-targeting gRNA) produced eGFP− cells (FIG. 12C and FIG. 12D). However, PCR for the expected eGFP− transposon junction in genomic DNA showed no evidence of targeted transposition in any of the transfections, suggesting that the eGFP− cells had lost eGFP expression by another mechanism (FIG. 12E). Although no targeted transposition by Himar-dCas9 into a genomic locus was observed here, an optimized mammalian testbed may enable screening for site-specific transposition events among larger samples of transposon insertions and shed light on the determinants of site-specific transposition in mammalian cells.

TABLE 1
Plasmids used in this study.

| | Origin of | Size | | | |
|---|---|---|---|---|---|
| Plasmid | replication | (bp) | Selection | Features | Purpose |
| pET- | ROP | 10864 | carb | 6xHis tag, T7 | HdCas9 protein |
| Himar- | | | | promoter | purification |
| dCasS | | | | | |
| pGT-B1 | pBBR1 | 6235 | carb | constitutive sfGFP | target plasmid for in |
| | | | | gene | vitro assays |
| pHimar6 | R6K | 3394 | kan | Himar transposon | Himar transposon |
| | | | | with chlor resistance | donor plasmid for in |
| | | | | cassette, RP4 oriT | vitro and E. coli in |
| | | | | | vivo assays |
| pTarget | ColE1 | 3237 | spec | constitutive sfGFP | target plasmid for |
| | | | | gene | E. coli in vivo assays |
| pHimar1C9 | p15A | 3846 | carb | Himar1C9 on tet- | bacterial expression |
| | | | | inducible promoter | vector for Himar1C9 |
| pHdCas9- | p15A | 8200 | carb | Himar-dCas9 on tet- | bacterial expression |
| gRNA1 | | | | inducible promoter, | vector for Himar- |
| | | | | constitutively | dCas9 and gRNA_1 |
| | | | | expressed gRNA_1 | |
| pHdCas9- | p15A | 8200 | carb | Himar-dCas9 on tet- | bacterial expression |
| gRNA4 | | | | inducible promoter, | vector for Himar- |
| | | | | constitutively | dCas9 and gRNA_4 |
| | | | | expressed gRNA_4 | |
| pHdCas9- | p15A | 8200 | carb | Himar-dCas9 on tet- | bacterial expression |
| gRNA5 | | | | inducible promoter, | vector for Himar- |
| | | | | constitutively | dCas9 and gRNA_5 |
| | | | | expressed gRNA_5 | |
| pHdCas9 | p15A | 7738 | carb | Himar-dCas9 on tet- | bacterial expression |
| | | | | inducible promoter | vector for Himar- |
| | | | | | dCas9 |
| pdCas9- | p15A | 6847 | carb | dCas9 on tet- | bacterial expression |
| carb | | | | inducible promoter | vector for Himar- |
| | | | | | dCas9 |
| pHdCas9- | p15A | 8191 | chlor | Himar-dCas9 on tet- | bacterial expression |
| gRNA5- | | | | inducible promoter, | vector for Himar- |
| gRNA16 | | | | constitutively | dCas9, gRNA_5, |
| | | | | expressed gRNA_5 | gRNA_16 |
| | | | | and gRNA_16 | |
| pdCas9- | p15A | 7099 | chlor | dCas9 on tet- | bacterial expression |
| gRNA5- | | | | inducible promoter, | vector for dCas9, |
| gRNA16 | | | | constitutively | gRNA_5, gRNA_16 |
| | | | | expressed gRNA_5 | |
| | | | | and gRNA_16 | |

TABLE 2
gRNA sequence used in this study

| | | | Target | Spacing | |
|---|---|---|---|---|---|
| gRNA | | Target | strand | to TA | SEQ ID |
| name | Sequence | gene | (T/N) | site (bp) | NO: |
| gRNA_1 | GTCGTTACCAGAGTCGGCCA | sfGFP | N | 8 | 17 |
| gRNA_2 | TCAGTGCTTTGCTCGTTATC | sfGFP | T | 7 | 18 |
| gRNA_3 | CGTTCCTGCACATAGCCTTC | sfGFP | N | 13 | 19 |
| gRNA_4 | CGGCACGTACAAAACGCGTG | sfGFP | T | 8 | 20 |
| gRNA_5 | GTCGGCGGGGTGCTTCACGT | mCherry | N | 10 | 21 |
| gRNA_7 | ACCAGAGTCGGCCAAGGTAC | sfGFP | N | 14 | 22 |
| gRNA_8 | CTGCACATAGCCTTCCGGCA | sfGFP | N | 18 | 23 |
| gRNA_9 | CAATGCCTTTCAGCTCAATG | sfGFP | N | 5 | 24 |
| gRNA_10 | CAGCTCAATGCGGTTTACCA | sfGFP | N | 15 | 25 |
| gRNA_11 | GTAAACCGCATTGAGCTGAA | sfGFP | T | 6 | 26 |
| gRNA_12 | CAATATCCTGGGCCATAAGC | sfGFP | T | 11 | 27 |
| gRNA_13 | AGAACAGGACCATCACCGAT | sfGFP | N | 17 | 28 |
| gRNA_14 | GTGCTCAGATAGTGATTGTC | sfGFP | N | 16 | 29 |
| gRNA_15 | GAACTGGATGGTGATGTCAA | sfGFP | T | 9 | 30 |
| gRNA_16 | CCTTCCCCGAGGGCTTCAAG | mCherry | T | 12 | 31 |
| gRNA_18 | ACGCGATCACATGGTTCTGC | sfGFP | T | 17 | 32 |

T Indicates that the gRNA is complementary to the Template strand of the gene, while N indicates that the gRNA complements the Non-template strand. gRNAs that target the same TA insertion site are labeled with the same color. gRNAs 11, 13, and 15 all target different sites uniquely.

TABLE 3
Oligonucleotides used in this study.

| | | | Tm | | SEQ |
|---|---|---|---|---|---|
| Name | Sequence (5′-3′) | Target | (° C.) | Function | ID NO: |
| p433 | CGCTTACAAT | pGT-B1 | 67 | qPCR for Himar | 33 |
| | TTCCATTCGC | | | transposon pGT-B1 | |
| | CATTC | | | junction | |
| p415 | CCCTGCAAAG | pHimar6 | 71 | qPCR for Himar | 34 |
| | CCCCTCTTTA | transposon | | transposon pGT-B1 | |
| | CG | | | junction | |
| p828 | CTGCGCAACC | pGT-B1 | 70 | Control qPCR for pGT-B1 | 35 |
| | CAAGTGCTAC | | | | |
| p829 | CAGTCCAGA | pGT-B1 | 67 | Control qPCR for pGT-B1 | 36 |
| | GAAATCGGC | | | | |
| | ATTCA | | | | |
| p923 | Biotin/GCCATA | pHimar6 | 68 | In vitro transposon | 37 |
| | AACTGCCAG | transposon | | sequencing library | |
| | GCATCAA | | | preparation | |
| p922 | CCTTCTTGCG | pGT-B1 | 67 | In vitro transposon | 38 |
| | CATCTCACG | | | sequencing library | |
| | | | | preparation | |
| Adapter_T | Phosphate/AGA | | | Anneal to make Y-shaped | 39 |
| | TCGGAAGAG | | | adapter for Tn-seq library | |
| | CACACGTCTG | | | prep | |
| | AACTCCAGTC | | | | |
| | AC | | | | |
| Adapter_B | GTCTCGTGG | | | Anneal to make Y-shaped | 40 |
| | GCTCGGGCT | | | adapter for Tn-seq library | |
| | CTTCCGATCT | | | prep | |
| | *N*N | | | | |
| p790 | AATGATACGG | Himar | 73 | Add barcode & P5 | 41 |
| | CGACCACCG | transposon | | sequence to Himar | |
| | AGATCTacacT | IR | | transposon ends for | |
| | AGATCGCCG | | | Illumina sequencing | |
| | CCagaccggggact | | | | |
| | tatcatccaacctgt | | | | |
| p791 | AATGATACGG | Himar | 73 | Add barcode & P5 | 42 |
| | CGACCACCG | transposon | | sequence to Himar | |
| | AGATCTacacC | IR | | transposon ends for | |
| | TCTCTATCGC | | | Illumina sequencing | |
| | Cagaccggggactat | | | | |
| | catccaacctgt | | | | |
| p792 | AATGATACGG | Himar | 73 | Add barcode & P5 | 43 |
| | CGACCACCG | transposon | | sequence to Himar | |
| | AGATCTacacT | IR | | transposon ends for | |
| | ATCCTCTCGC | | | Illumina sequencing | |
| | Cagaccggggactta | | | | |
| | tcatccaacctgt | | | | |
| 44p793 | AATGATACGG | Himar | 73 | Add barcode & P5 | 44 |
| | CGACCACCG | transposon | | sequence to Himar | |
| | AGATCTacacA | IR | | transposon ends for | |
| | GAGTAGACG | | | Illumina sequencing | |
| | CCagaccggggact | | | | |
| | tatcatccaacctgt | | | | |
| p74594 | AATGATACGG | Himar | 73 | Add barcode & P5 | 45 |
| | CGACCACCG | transposon | | sequence to Himar | |
| | AGATCTacacG | IR | | transposon ends for | |
| | TAAGGAGCG | | | Illumina sequencing | |
| | CCagaccggggact | | | | |
| | tatcatccaacctgt | | | | |
| p795 | AATGATACGG | Himar | 73 | Add barcode & P5 | 46 |
| | CGACCACCG | transposon | | sequence to Himar | |
| | AGATCTacacA | IR | | transposon ends for | |
| | CTGCATACGC | | | Illumina sequencing | |
| | Cagaccggggactta | | | | |
| | tcatccaacctgt | | | | |
| p712 | CGCCagaccggg | Himar | 67 | Read 1 primer for Illumina | 47 |
| | gacttatcatccaacct | transposon | | sequencing | |
| | gt | IR | | | |
| p713 | CGGAAGAGC | Himar | 67 | Index 1 primer for | 48 |
| | CCGAGCCCA | sequencing | | Illumina sequencing | |
| | CGAGAC | library | | | |
| p898 | TTTGAGTGAG | ColE1 oriR | 67 | qPCR for Himar | 49 |
| | CTGATACCGC | | | transposon-plasmid | |
| | TC | | | junctions in pTarget | |
| | | | | plasmid | |
| p899 | GAGCGGTAT | ColE1 oriR | 67 | Control qPCR for pTarget | 50 |
| | CAGCTCACTC | | | | |
| | AAA | | | | |
| p900 | TCCCTTAACG | ColE1 oriR | 67 | Control qPCR for pTarget | 51 |
| | TGAGTTTTCG | | | | |
| | TTCC | | | | |

### Example 7: Sequences

Unless otherwise stated, nucleic acid sequences in the text of this specification and SEQ ID number listing, are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent. Also, a given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA:DNA duplexes (hybridized strands), base-pairs are adenine:thymine or guanine:cytosine; for DNA:RNA duplexes, base-pairs are adenine:uracil or guanine:cytosine. Thus, the nucleotide sequence of a blunt-ended double-stranded polynucleotide that is perfectly hybridized (where there is “100% complementarity” between the strands or where the strands are “complementary”) is unambiguously defined by providing the nucleotide sequence of one strand, whether given as DNA or RNA.

Himar1 WT
(SEQ ID NO: 1)
MEKKEFRVLIKYCFLKGKNTVEAKTWLDNEFPDSAPGKSTIIDWYAKFKRGEMSTEDGE
RSGRPKEVVTDENIKKIHKMILNDRKMKLIEIAEALKISKERVGHIIHQYLDMRKLCAKW
VPRELTFDQKQQRVDDSERCLQLLTRNTPEFFRRYVTMDETWLHHYTPESNRQSAEWT
ATGEPSPKRGKTQKSAGKVMASVFWDAHGIIFIDYLEKGKTINSDYYMALLERLKVEIA
AKRPHMKKKKVLFHQDNAPCHKSLRTMAKIHELGFELLPHPPYSPDLAPSDFFLFSDLK
RMLAGKKFGCNEEVIAETEAYFEAKPKEYYQNGIKKLEGRYNRCIALEGNYVE
Himar1C9
(SEQ ID NO: 2)
MEKKEFRVLIKYCFLKGKNTVEAKTWLDNEFPDSAPGKSTIIDWYAKFKRGEMSTEDGE
RSGRPKEVVTDENIKKIHKMILNDRKMKLIEIAEALKISKERVGHIIHQYLDMRKLCAKW
VPRELTFDQKQRRVDDSKRCLQLLTRNTPEFFRRYVTMDETWLHHYTPESNRQSAEWT
ATGEPSPKRGKTQKSAGKVMASVFWDAHGIIFIDYLEKGKTINSDYYMALLERLKVEIA
AKRPHMKKKKVLFHQDNAPCHKSLRTMAKIHELGFELLPHPPYSPDLAPSDFFLFSDLK
RMLAGKKFGCNEEVIAETEAYFEAKPKEYYQNGIKKLEGRYNRCIALEGNYVE
Himar1C9-dCas9 fusion protein
(SEQ ID NO: 3)
MEKKEFRVLIKYCFLKGKNTVEAKTWLDNEFPDSAPGKSTIIDWYAKFKRGEMSTEDGE
RSGRPKEVVTDENIKKIHKMILNDRKMKLIEIAEALKISKERVGHIIHQYLDMRKLCAKW
VPRELTFDQKQRRVDDSKRCLQLLTRNTPEFFRRYVTMDETWLHHYTPESNRQSAEWT
ATGEPSPKRGKTQKSAGKVMASVFWDAHGIIFIDYLEKGKTINSDYYMALLERLKVEIA
AKRPHMKKKKVLFHQDNAPCHKSLRTMAKIHELGFELLPHPPYSPDLAPSDFFLFSDLK
RMLAGKKFGCNEEVIAETEAYFEAKPKEYYQNGIKKLEGRYNRCIALEGNYVESGSETP
GTSESATPESMDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLI
EGDLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQ
ELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFEKTEITLANGEIRKRPL
IETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYETRID
LSQLGGD
Hyperactive Tn5 transposase
(SEQ ID NO: 4)
MITSALHRAADWAKSVFSSAALGDPRRTARLVNVAAQLAKYSGKSITISSEGSKAAQEG
AYRFIRNPNVSAEAIRKAGAMQTVKLAQEFPELLAIEDTTSLSYRHQVAEELGKLGSIQD
KSRGWWVHSVLLLEATTFRTVGLLHQEWWMRPDDPADADEKESGKWLAAAATSRLR
MGSMMSNVIAVCDREADIHAYLQDKLAHNERFVVRSKHPRKDVESGLYLYDHLKNQP
ELGGYQISIPQKGVVDKRGKRKNRPARKASLSLRSGRITLKQGNITLNAVLAEEINPPKG
ETPLKWLLLTSEPVESLAQALRVIDIYTHRWRIEEFHKAWKTGAGAERQRMEEPDNLER
MVSILSFVAVRLLQLRESFTPPQALRAQGLLKEAEHVESQSAETVLTPDECQLLGYLDK
GKRKRKEKAGSLQWAYMAIARLGGFMDSKRTGIASWGALWEGWEALQSKLDGFLAA
KDLMAQGIKI
Tn5-dCas9 fusion protein with XTEN linker
(SEQ ID NO: 5)
MITSALHRAADWAKSVFSSAALGDPRRTARLVNVAAQLAKYSGKSITISSEGSKAAQEG
AYRFIRNPNVSAEAIRKAGAMQTVKLAQEFPELLAIEDTTSLSYRHQVAEELGKLGSIQD
KSRGWWVHSVLLLEATTFRTVGLLHQEWWMRPDDPADADEKESGKWLAAAATSRLR
MGSMMSNVIAVCDREADIHAYLQDKLAHNERFVVRSKHPRKDVESGLYLYDHLKNQP
ELGGYQISIPQKGVVDKRGKRKNRPARKASLSLRSGRITLKQGNITLNAVLAEEINPPKG
ETPLKWLLLTSEPVESLAQALRVIDIYTHRWRIEEFHKAWKTGAGAERQRMEEPDNLER
MVSILSFVAVRLLQLRESFTPPQALRAQGLLKEAEHVESQSAETVLTPDECQLLGYLDK
GKRKRKEKAGSLQWAYMAIARLGGFMDSKRTGIASWGALWEGWEALQSKLDGFLAA
KDLMAQGIKISGSETPGTSESATPESMDKKYSIGLAIGTNSVGWAVITDEYKVPSKKFKV
LGNTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDD
LDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNV
PSEEVVKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLDAT
LIHQSITGLYETRIDLSQLGGD
dCas9 (D10A, H840A)
(SEQ ID NO: 6)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
Himar1C9-dCas9 fusion protein with N-terminus 3xFLAG and SV40
mammalian NLS
(SEQ ID NO: 7)
MDYKDHDGDYKDHDIDYKDDDDKMAPKKKRKVGIHRGVPGGSGSMEKKEFRVLIKY
CFLKGKNTVEAKTWLDNEFPDSAPGKSTIIDWYAKFKRGEMSTEDGERSGRPKEVVTD
ENIKKIHKMILNDRKMKLIEIAEALKISKERVGHIIHQYLDMRKLCAKWVPRELTFDQKQ
RRVDDSKRCLQLLTRNTPEFFRRYVTMDETWLHHYTPESNRQSAEWTATGEPSPKRGK
TQKSAGKVMASVFWDAHGIIFIDYLEKGKTINSDYYMALLERLKVEIAAKRPHMKKKK
VLFHQDNAPCHKSLRTMAKIHELGFELLPHPPYSPDLAPSDFFLFSDLKRMLAGKKFGC
NEEVIAETEAYFEAKPKEYYQNGIKKLEGRYNRCIALEGNYVESGSETPGTSESATPESD
KKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD
Himar1C9-dCas9 fusion protein with C-terminal E. coli SsrA
degradation tag
(SEQ ID NO: 8)
MEKKEFRVLIKYCFLKGKNTVEAKTWLDNEFPDSAPGKSTIIDWYAKFKRGEMSTEDGE
RSGRPKEVVTDENIKKIHKMILNDRKMKLIEIAEALKISKERVGHIIHQYLDMRKLCAKW
VPRELTFDQKQRRVDDSKRCLQLLTRNTPEFFRRYVTMDETWLHHYTPESNRQSAEWT
ATGEPSPKRGKTQKSAGKVMASVFWDAHGIIFIDYLEKGKTINSDYYMALLERLKVEIA
AKRPHMKKKKVLFHQDNAPCHKSLRTMAKIHELGFELLPHPPYSPDLAPSDFFLFSDLK
RMLAGKKFGCNEEVIAETEAYFEAKPKEYYQNGIKKLEGRYNRCIALEGNYVESGSETP
GTSESATPESMDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLI
EGDLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQ
ELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYETRID
LSQLGGDRPAANDENYALAA
Himar1 Transposon inverted repeat
(SEQ ID NO: 9)
ACAGGTTGGATGATAAGTCCCCGGTCT
Himar1 mini-transposon containing chloramphenicol resistance
cassette as payload (from plasmid pHimar6). Himar1 inverted
repeat sequences are bolded.
(SEQ ID NO: 10)
ACAGGTTGGATGATAAGTCCCCGGTCTTCGTATGCCGTCTTCTGCTTGGCGCGCCC
TCGAGCAATTGCCGACCGAATTTTTATGTCGTAAAGAGGGGCTTTGCAGGGGGTGGA
CTCAGAAAGATGAGAATAGATGACTATTGTAGTTGAAACACATAGAAAGTTGCTGA
TATACAGACCGATACGCATATCGGGATGAACCATGAGTACGTTCTTTTCTCAAAAAA
CATAAATATTCGAAAAGAGATGCAATAAATTAAGGAGAGGTTATACTCTAGAGTAG
TAGATTATTTTAGGAATTTAGATGTTTTGTATGAAATAGATGCTTCGTATGGAATTAA
TGAAATTTTTAGTCAGGTAAAAAAGGTAATAGGAGAATATTATGGAGAAAAAAATC
ACTGGATATACCACCGTTGATATATCCCAATGGCATCGTAAAGAACATTTTGAGGCA
TTTCAGTCAGTTGCTCAATGTACCTATAACCAGACCGTTCAGCTGGATATTACGGCC
TTTTTAAAGACCGTAAAGAAAAATAAGCACAAGTTTTATCCGGCCTTTATTCACATT
CTTGCCCGCCTGATGAATGCTCATCCGGAATTTCGTATGGCAATGAAAGACGGTGAG
CTGGTGATATGGGATAGTGTTCACCCTTGTTACACCGTTTTCCATGAGCAAACTGAA
ACGTTTTCATCGCTCTGGAGTGAATACCACGACGATTTCCGGCAGTTTCTACACATA
TATTCGCAAGATGTGGCGTGTTACGGTGAAAACCTGGCCTATTTCCCTAAAGGGTTT
ATTGAGAATATGTTTTTCGTCTCAGCCAATCCCTGGGTGAGTTTCACCAGTTTTGATT
TAAACGTGGCCAATATGGACAACTTCTTCGCCCCCGTTTTCACCATGGGCAAATATT
ATACGCAAGGCGACAAGGTGCTGATGCCGCTGGCGATTCAGGTTCATCATGCCGTTT
GTGATGGCTTCCATGTCGGCAGAATGCTTAATGAATTACAACAGTACTGCGATGAGT
GGCAGGGCGGGGCGTAAAAACAATAGGCCACATGCAACTGTCTAGAATGCGAGAGT
AGGGAACTGCCAGGCATCAAATAAAACGAAAGGCTCAGTCGAAAGACTGGGCCTTT
CGTTTTATCTGTTGTTTGTCGGTGAACGCTCTCCTGAGTAGGACAAATCCGCCGGGA
GCGGATTTGAACGTTGCGAAGCAACGGCCCGGAGGGTGGCGGGCAGGACGCCCGCC
ATAAACTGCCAGGCATCAAATTAAGCAGAAGGCCATCCTGACGGATGGCCTTTTTGC
GTTTCTACCTGCAGGGCGCGCCAAGCAGAAGACGGCATACGAAGACCGGGGACTT
ATCATCCAACCTGT
DNA coding sequence for Himar1C9-dCas9 fusion protein
with XTEN linker
(SEQ ID NO: 11)
ATGGAAAAAAAGGAATTTCGTGTTTTGATAAAATACTGTTTTCTGAAGGGAAAAAAT
ACAGTGGAAGCAAAAACTTGGCTTGATAATGAGTTTCCGGACTCTGCCCCAGGGAA
ATCAACAATAATTGATTGGTATGCAAAATTCAAGCGTGGTGAAATGAGCACGGAGG
ACGGTGAACGCAGTGGACGCCCGAAAGAGGTGGTTACCGACGAAAACATCAAAAA
AATCCACAAAATGATTTTGAATGACCGTAAAATGAAGTTGATCGAGATAGCAGAGG
CCTTAAAGATATCAAAGGAACGTGTTGGTCATATCATTCATCAATATTTGGATATGC
GGAAGCTCTGTGCGAAATGGGTGCCGCGCGAGCTCACATTTGACCAAAAACAACGA
CGTGTTGATGATTCTAAGCGGTGTTTGCAGCTGTTAACTCGTAATACACCCGAGTTTT
TCCGTCGATATGTGACAATGGATGAAACATGGCTCCATCACTACACTCCTGAGTCCA
ATCGACAGTCGGCTGAGTGGACAGCGACCGGTGAACCGTCTCCGAAGCGTGGAAAG
ACTCAAAAGTCCGCTGGCAAAGTAATGGCCTCTGTTTTTTGGGATGCGCATGGAATA
ATTTTTATCGATTATCTTGAGAAGGGAAAAACCATCAACAGTGACTATTATATGGCG
TTATTGGAGCGTTTGAAGGTCGAAATCGCGGCAAAACGGCCCCACATGAAGAAGAA
AAAAGTGTTGTTCCACCAAGACAACGCACCGTGCCACAAGTCATTGAGAACGATGG
CAAAAATTCATGAATTGGGCTTCGAATTGCTTCCCCACCCGCCGTATTCTCCAGATCT
GGCCCCCAGCGACTTTTTCTTGTTCTCAGACCTCAAAAGGATGCTCGCAGGGAAAAA
ATTTGGCTGCAATGAAGAGGTGATCGCCGAAACTGAGGCCTATTTTGAGGCAAAAC
CGAAGGAGTACTACCAAAATGGTATCAAAAAATTGGAAGGTCGTTATAATCGTTGT
ATCGCTCTTGAAGGGAACTATGTTGAAAGCGGTTCCGAAACTCCCGGTACATCAGAA
AGCGCGACCCCCGAAAGCATGGATAAAAAGTATTCTATTGGTTTAGCTATCGGCACA
AATAGCGTCGGATGGGCGGTGATCACTGATGAATATAAGGTTCCGTCTAAAAAGTTC
AAGGTTCTGGGAAATACAGACCGCCACAGTATCAAAAAAAATCTTATAGGGGCTCT
TTTATTTGACAGTGGAGAGACAGCGGAAGCGACTCGTCTCAAACGGACAGCTCGTA
GAAGGTATACACGTCGGAAGAATCGTATTTGTTATCTACAGGAGATTTTTTCAAATG
AGATGGCGAAAGTAGATGATAGTTTCTTTCATCGACTTGAAGAGTCTTTTTTGGTGG
AAGAAGACAAGAAGCATGAACGTCATCCTATTTTTGGAAATATAGTAGATGAAGTT
GCTTATCATGAGAAATATCCAACTATCTATCATCTGCGAAAAAAATTGGTAGATTCT
ACTGATAAAGCGGATTTGCGCTTAATCTATTTGGCCTTAGCGCATATGATTAAGTTTC
GTGGTCATTTTTTGATTGAGGGAGATTTAAATCCTGATAATAGTGATGTGGACAAAC
TATTTATCCAGTTGGTACAAACCTACAATCAATTATTTGAAGAAAACCCTATTAACG
CAAGTGGAGTAGATGCTAAAGCGATTCTTTCTGCACGATTGAGTAAATCAAGACGAT
TAGAAAATCTCATTGCTCAGCTCCCCGGTGAGAAGAAAAATGGCTTATTTGGGAATC
TCATTGCTTTGTCATTGGGTTTGACCCCTAATTTTAAATCAAATTTTGATTTGGCAGA
AGATGCTAAATTACAGCTTTCAAAAGATACTTACGATGATGATTTAGATAATTTATT
GGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCT
ATCAGCTTCAATGATTAAACGCTACGATGAACATCATCAAGACTTGACTCTTTTAAA
AGCTTTAGTTCGACAACAACTTCCAGAAAAGTATAAAGAAATCTTTTTTGATCAATC
AAAAAACGGATATGCAGGTTATATTGATGGGGGAGCTAGCCAAGAAGAATTTTATA
AATTTATCAAACCAATTTTAGAAAAAATGGATGGTACTGAGGAATTATTGGTGAAAC
TAAATCGTGAAGATTTGCTGCGCAAGCAACGGACCTTTGACAACGGCTCTATTCCCC
ATCAAATTCACTTGGGTGAGCTGCATGCTATTTTGAGAAGACAAGAAGACTTTTATC
CATTTTTAAAAGACAATCGTGAGAAGATTGAAAAAATCTTGACTTTTCGAATTCCTT
ATTATGTTGGTCCATTGGCGCGTGGCAATAGTCGTTTTGCATGGATGACTCGGAAGT
CTGAAGAAACAATTACCCCATGGAATTTTGAAGAAGTTGTCGATAAAGGTGCTTCAG
CTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAA
AGGTCAAATATGTTACTGAAGGAATGCGAAAACCAGCATTTCTTTCAGGTGAACAG
AAGAAAGCCATTGTTGATTTACTCTTCAAAACAAATCGAAAAGTAACCGTTAAGCA
ATTAAAAGAAGATTATTTCAAAAAAATAGAATGTTTTGATAGTGTTGAAATTTCAGG
AGTTGAAGATAGATTTAATGCTTCATTAGGTACCTACCATGATTTGCTAAAAATTAT
TAAAGATAAAGATTTTTTGGATAATGAAGAAAATGAAGATATCTTAGAGGATATTGT
TTTAACATTGACCTTATTTGAAGATAGGGAGATGATTGAGGAAAGACTTAAAACATA
TGCTCACCTCTTTGATGATAAGGTGATGAAACAGCTTAAACGTCGCCGTTATACTGG
TTGGGGACGTTTGTCTCGAAAATTGATTAATGGTATTAGGGATAAGCAATCTGGCAA
AACAATATTAGATTTTTTGAAATCAGATGGTTTTGCCAATCGCAATTTTATGCAGCTG
ATCCATGATGATAGTTTGACATTTAAAGAAGACATTCAAAAAGCACAAGTGTCTGG
ACAAGGCGATAGTTTACATGAACATATTGCAAATTTAGCTGGTAGCCCTGCTATTAA
AAAAGGTATTTTACAGACTGTAAAAGTTGTTGATGAATTGGTCAAAGTAATGGGGC
GGCATAAGCCAGAAAATATCGTTATTGAAATGGCACGTGAAAATCAGACAACTCAA
AAGGGCCAGAAAAATTCGCGAGAGCGTATGAAACGAATCGAAGAAGGTATCAAAG
AATTAGGAAGTCAGATTCTTAAAGAGCATCCTGTTGAAAATACTCAATTGCAAAATG
AAAAGCTCTATCTCTATTATCTCCAAAATGGAAGAGACATGTATGTGGACCAAGAAT
TAGATATTAATCGTTTAAGTGATTATGATGTCGATGCCATTGTTCCACAAAGTTTCCT
TAAAGACGATTCAATAGACAATAAGGTCTTAACGCGTTCTGATAAAAATCGTGGTA
AATCGGATAACGTTCCAAGTGAAGAAGTAGTCAAAAAGATGAAAAACTATTGGAGA
CAACTTCTAAACGCCAAGTTAATCACTCAACGTAAGTTTGATAATTTAACGAAAGCT
GAACGTGGAGGTTTGAGTGAACTTGATAAAGCTGGTTTTATCAAACGCCAATTGGTT
GAAACTCGCCAAATCACTAAGCATGTGGCACAAATTTTGGATAGTCGCATGAATACT
AAATACGATGAAAATGATAAACTTATTCGAGAGGTTAAAGTGATTACCTTAAAATCT
AAATTAGTTTCTGACTTCCGAAAAGATTTCCAATTCTATAAAGTACGTGAGATTAAC
AATTACCATCATGCCCATGATGCGTATCTAAATGCCGTCGTTGGAACTGCTTTGATT
AAGAAATATCCAAAACTTGAATCGGAGTTTGTCTATGGTGATTATAAAGTTTATGAT
GTTCGTAAAATGATTGCTAAGTCTGAGCAAGAAATAGGCAAAGCAACCGCAAAATA
TTTCTTTTACTCTAATATCATGAACTTCTTCAAAACAGAAATTACACTTGCAAATGGA
GAGATTCGCAAACGCCCTCTAATCGAAACTAATGGGGAAACTGGAGAAATTGTCTG
GGATAAAGGGCGAGATTTTGCCACAGTGCGCAAAGTATTGTCCATGCCCCAAGTCA
ATATTGTCAAGAAAACAGAAGTACAGACAGGCGGATTCTCCAAGGAGTCAATTTTA
CCAAAAAGAAATTCGGACAAGCTTATTGCTCGTAAAAAAGACTGGGATCCAAAAAA
ATATGGTGGTTTTGATAGTCCAACGGTAGCTTATTCAGTCCTAGTGGTTGCTAAGGT
GGAAAAAGGGAAATCGAAGAAGTTAAAATCCGTTAAAGAGTTACTAGGGATCACAA
TTATGGAAAGAAGTTCCTTTGAAAAAAATCCGATTGACTTTTTAGAAGCTAAAGGAT
ATAAGGAAGTTAAAAAAGACTTAATCATTAAACTACCTAAATATAGTCTTTTTGAGT
TAGAAAACGGTCGTAAACGGATGCTGGCTAGTGCCGGAGAATTACAAAAAGGAAAT
GAGCTGGCTCTGCCAAGCAAATATGTGAATTTTTTATATTTAGCTAGTCATTATGAA
AAGTTGAAGGGTAGTCCAGAAGATAACGAACAAAAACAATTGTTTGTGGAGCAGCA
TAAGCATTATTTAGATGAGATTATTGAGCAAATCAGTGAATTTTCTAAGCGTGTTAT
TTTAGCAGATGCCAATTTAGATAAAGTTCTTAGTGCATATAACAAACATAGAGACAA
ACCAATACGTGAACAAGCAGAAAATATTATTCATTTATTTACGTTGACGAATCTTGG
AGCTCCCGCTGCTTTTAAATATTTTGATACAACAATTGATCGTAAACGATATACGTCT
ACAAAAGAAGTTTTAGATGCCACTCTTATCCATCAATCCATCACTGGTCTTTATGAA
ACACGCATTGATTTGAGTCAGCTAGGAGGTGACTAA
Tn5 transposon inverted repeat
(SEQ ID NO: 12)
CTGTCTCTTATACACATCT
Tn5 mini-transposon containing chloramphenicol resistance
cassette as payload. Tn5 inverted repeat sequences are bolded
(SEQ ID NO: 13)
CTGTCTCTTATACACATCTCAACCATCATCGATGAATTTTCTCGGGTGTTCTCGCAT
ATTGGCTCGAATTCCTGCAGCCCCTCTAGAGTAGTAGATTATTTTAGGAATTTAGAT
GTTTTGTATGAAATAGATGCTTCGTATGGAATTAATGAAATTTTTAGTCAGGTAAAA
AAGGTAATAGGAGAATATTATGGAGAAAAAAATCACTGGATATACCACCGTTGATA
TATCCCAATGGCATCGTAAAGAACATTTTGAGGCATTTCAGTCAGTTGCTCAATGTA
CCTATAACCAGACCGTTCAGCTGGATATTACGGCCTTTTTAAAGACCGTAAAGAAAA
ATAAGCACAAGTTTTATCCGGCCTTTATTCACATTCTTGCCCGCCTGATGAATGCTCA
TCCGGAATTTCGTATGGCAATGAAAGACGGTGAGCTGGTGATATGGGATAGTGTTCA
CCCTTGTTACACCGTTTTCCATGAGCAAACTGAAACGTTTTCATCGCTCTGGAGTGA
ATACCACGACGATTTCCGGCAGTTTCTACACATATATTCGCAAGATGTGGCGTGTTA
CGGTGAAAACCTGGCCTATTTCCCTAAAGGGTTTATTGAGAATATGTTTTTCGTCTCA
GCCAATCCCTGGGTGAGTTTCACCAGTTTTGATTTAAACGTGGCCAATATGGACAAC
TTCTTCGCCCCCGTTTTCACCATGGGCAAATATTATACGCAAGGCGACAAGGTGCTG
ATGCCGCTGGCGATTCAGGTTCATCATGCCGTTTGTGATGGCTTCCATGTCGGCAGA
ATGCTTAATGAATTACAACAGTACTGCGATGAGTGGCAGGGCGGGGCGTAAAAACA
ATAGGCCACATGCAACTGTCTAGAATGCGAGAGTAGGGAACTGCCAGGCATCAAAT
AAAACGAAAGGCTCAGTCGAAAGACTGGGCCTTTCGTTTTATTGAACGGTAGCATCT
TGACGACGCAGCTTGCCAACGACTACGCACTAGCCAACAAGAGCTTCAGGGTTGAG
ATGTGTATAAGAGACAG

### REFERENCES

1. Esvelt K M, Wang H H. Genome-scale engineering for systems and synthetic biology. Mol Syst Biol 2013; 9:641. DOI: 10.1038/msb.2012.66. Crossref, Medline, Google Scholar
2. Andrews B J, Proteau G A, Beatty L G, et al. The FLP recombinase of the 2 micron circle DNA of yeast: interaction with its target sequences. Cell 1985; 40:795-803. DOI: 10.1016/0092-8674(85)90339-3. Crossref, Medline, Google Scholar
3. Abremski K, Hoess R. Bacteriophage P1 site-specific recombination. Purification and properties of the Cre recombinase protein. J Biol Chem 1984; 259:1509-1514. Medline, Google Scholar
4. Bolusani S, Ma C H, Paek A, et al. Evolution of variants of yeast site-specific recombinase Flp that utilize native genomic sequences as recombination target sites. Nucleic Acids Res 2006; 34:5259-5269. DOI: 10.1093/nar/gk1548. Crossref, Medline, Google Scholar
5. Buchholz F, Stewart A F. Alteration of Cre recombinase site specificity by substrate-linked protein evolution. Nat Biotechnol 2001; 19:1047-1052. DOI: 10.1038/nbt1101-1047. Crossref, Medline, Google Scholar
6. Cong L, Ran F A, Cox D, et al. Multiplex genome engineering using CRISPR/Cas systems. Science 2013; 339:819-823. DOI: 10.1126/science.1231143. Crossref, Medline, Google Scholar
7. Jinek M, Chylinski K, Fonfara I, et al. A programmable dual-RNA-guided DNA endonuclease in adaptive bacterial immunity. Science 2012; 337:816-821. DOI: 10.1126/science.1225829. Crossref, Medline, Google Scholar
8. Urnov F D, Rebar E J, Holmes M C, et al. Genome editing with engineered zinc finger nucleases. Nat Rev Genet 2010; 11:636-646. DOI: 10.1038/nrg2842. Crossref, Medline, Google Scholar
9. Joung J K, Sander J D. TALENs: a widely applicable technology for targeted genome editing. Nat Rev Mol Cell Biol 2013; 14:49-55. DOI: 10.1038/nrm3486. Crossref, Medline, Google Scholar
10. Kowalczykowski S C. An overview of the molecular mechanisms of recombinational DNA repair. Cold Spring Harb Perspect Biol 2015; 7. DOI: 10.1101/cshperspect.a016410. Google Scholar
11. Munoz-Lopez M, Garcia-Perez J L. DNA transposons: nature and applications in genomics. Curr Genomics 2010; 11:115-128. DOI: 10.2174/138920210790886871. Crossref, Medline, Google Scholar
12. Curcio M J, Derbyshire K M. The outs and ins of transposition: from mu to kangaroo. Nat Rev Mol Cell Biol 2003; 4:865-877. DOI: 10.1038/nrm1241. Crossref, Medline, Google Scholar
13. Lampe D J, Churchill M E, Robertson H M. A purified mariner transposase is sufficient to mediate transposition in vitro. EMBO J 1996; 15:5470-5479. DOI: 10.1002/j.1460-2075.1996.tb00930.x. Crossref, Medline, Google Scholar
14. Richardson J M, Dawson A, O'Hagan N, et al. Mechanism of Mos1 transposition: insights from structural analysis. EMBO J 2006; 25:1324-1334. DOI: 10.1038/sj.emboj.7601018. Crossref, Medline, Google Scholar
15. Richardson J M, Colloms S D, Finnegan D J, et al. Molecular architecture of the Mos1 paired-end complex: the structural basis of DNA transposition in a eukaryote. Cell 2009; 138:1096-1108. DOI: 10.1016/j.cell.2009.07.012. Crossref, Medline, Google Scholar
16. Claeys Bouuaert C, Lipkow K, Andrews S S, et al. The autoregulation of a eukaryotic DNA transposon. eLife 2013; 2:e00668. DOI: 10.7554/eLife.00668. Crossref, Medline, Google Scholar
17. van Opijnen T, Camilli A. Transposon insertion sequencing: a new tool for systems-level analysis of microorganisms. Nat Rev Microbiol 2013; 11:435-442. DOI: 10.1038/nrmicro3033. Crossref, Medline, Google Scholar
18. Zhang L, Sankar U, Lampe D J, et al. The Himar1 mariner transposase cloned in a recombinant adenovirus vector is functional in mammalian cells. Nucleic Acids Res 1998; 26:3687-3693. DOI: 10.1093/nar/26.16.3687. Crossref, Medline, Google Scholar
19. Lampe D J, Grant T E, Robertson H M. Factors affecting transposition of the Himar1 mariner transposon in vitro. Genetics 1998; 149:179-187. Medline, Google Scholar
20. Lampe D J, Akerley B J, Rubin E J, et al. Hyperactive transposase mutants of the Himar1 mariner transposon. Proc Natl Acad Sci USA 1999; 96:11428-11433. DOI: 10.1073/pnas.96.20.11428. Crossref, Medline, Google Scholar
21. Goodman A L, McNulty N P, Zhao Y, et al. Identifying genetic determinants needed to establish a human gut symbiont in its habitat. Cell Host Microbe 2009; 6:279-289. DOI: 10.1016/j.chom.2009.08.003. Crossref, Medline, Google Scholar
22. van Opijnen T, Bodi K L, Camilli A. Tn-seq: high-throughput parallel sequencing for fitness and genetic interaction studies in microorganisms. Nat Methods 2009; 6:767-772. DOI: 10.1038/nmeth.1377. Crossref, Medline, Google Scholar
23. Zhang J K, Pritchett M A, Lampe D J, et al. In vivo transposon mutagenesis of the methanogenic archaeon Methanosarcina acetivorans C2A using a modified version of the insect mariner-family transposable element Himar1. Proc Natl Acad Sci USA 2000; 97:9665-9670. DOI: 10.1073/pnas.160272597. Crossref, Medline, Google Scholar
24. Morero N R, Zuliani C, Kumar B, et al. Targeting IS608 transposon integration to highly specific sequences by structure-based transposon engineering. Nucleic Acids Res 2018; 46:4152-4163. DOI: 10.1093/nar/gky235. Crossref, Medline, Google Scholar
25. Maragathavally K J, Kaminski J M, Coates C J. Chimeric Mos1 and piggyBac transposases result in site-directed integration. FASEB J 2006; 20:1880-1882. DOI: 10.1096/fj.05-5485fje. Crossref, Medline, Google Scholar
26. Owens J B, Urschitz J, Stoytchev I, et al. Chimeric piggyBac transposases for genomic targeting in human cells. Nucleic Acids Res 2012; 40:6978-6991. DOI: 10.1093/nar/gks309. Crossref, Medline, Google Scholar
27. Owens J B, Mauro D, Stoytchev I, et al. Transcription activator like effector (TALE)-directed piggyBac transposition in human cells. Nucleic Acids Res 2013; 41:9197-9207. DOI: 10.1093/nar/gkt677. Crossref, Medline, Google Scholar
28. Luo W, Galvan D L, Woodard L E, et al. Comparative analysis of chimeric ZFP-, TALE- and Cas9-piggyBac transposases for integration into a single locus in human cells. Nucleic Acids Res 2017; 45:8411-8422. DOI: 10.1093/nar/gkx572. Crossref, Medline, Google Scholar
29. Feng X, Bednarz A L, Colloms S D. Precise targeted integration by a chimaeric transposase zinc-finger fusion protein. Nucleic Acids Res 2010; 38:1204-1216. DOI: 10.1093/nar/gkp1068. Crossref, Medline, Google Scholar
30. Strecker J, Ladha A, Gardner Z, et al. RNA-guided DNA insertion with CRISPR-associated transposases. Science 2019; 365:48-53. DOI: 10.1126/science.aax9181. Crossref, Medline, Google Scholar
31. Klompe S E, Vo P L H, Halpin-Healy T S, et al. Transposon-encoded CRISPR-Cas systems direct RNA-guided DNA integration. Nature 2019; 571:219-225. DOI: 10.1038/s41586-019-1323-z. Crossref, Medline, Google Scholar
32. Qi L S, Larson M H, Gilbert L A, et al. Repurposing CRISPR as an RNA-guided platform for sequence-specific control of gene expression. Cell 2013; 152:1173-1183. DOI: 10.1016/j.cell.2013.02.022. Crossref, Medline, Google Scholar
33. Bikard D, Jiang W, Samai P, et al. Programmable repression and activation of bacterial gene expression using an engineered CRISPR-Cas system. Nucleic Acids Res 2013; 41:7429-7437. DOI: 10.1093/nar/gkt520. Crossref, Medline, Google Scholar
34. Gilbert L A, Larson M H, Morsut L, et al. CRISPR-mediated modular RNA-guided regulation of transcription in eukaryotes. Cell 2013; 154:442-451. DOI: 10.1016/j.cell.2013.06.044. Crossref, Medline, Google Scholar
35. Guilinger J P, Thompson D B, Liu D R. Fusion of catalytically inactive Cas9 to FokI nuclease improves the specificity of genome modification. Nat Biotechnol 2014; 32:577-582. DOI: 10.1038/nbt.2909. Crossref, Medline, Google Scholar
36. Tsai S Q, Wyvekens N, Khayter C, et al. Dimeric CRISPR RNA-guided FokI nucleases for highly specific genome editing. Nat Biotechnol 2014; 32:569-576. DOI: 10.1038/nbt.2908. Crossref, Medline, Google Scholar
37. Gaudelli N M, Komor A C, Rees H A, et al. Programmable base editing of A*T to G*C in genomic DNA without DNA cleavage. Nature 2017; 551:464-471. DOI: 10.1038/nature24644. Crossref, Medline, Google Scholar
38. Komor A C, Kim Y B, Packer M S, et al. Programmable editing of a target base in genomic DNA without double-stranded DNA cleavage. Nature 2016; 533:420-424. DOI: 10.1038/nature17946. Crossref, Medline, Google Scholar
39. Chaikind B, Bessen J L, Thompson D B, et al. A programmable Cas9-serine recombinase fusion protein that operates on DNA sequences in mammalian cells. Nucleic Acids Res 2016; 44:9758-9770. DOI: 10.1093/nar/gkw707. Medline, Google Scholar
40. Kearns N A, Pham H, Tabak B, et al. Functional annotation of native enhancers with a Cas9-histone demethylase fusion. Nat Methods 2015; 12:401-403. DOI: 10.1038/nmeth.3325. Crossref, Medline, Google Scholar
41. Hilton I B, D'Ippolito A M, Vockley C M, et al. Epigenome editing by a CRISPR-Cas9-based acetyltransferase activates genes from promoters and enhancers. Nat Biotechnol 2015; 33:510-517. DOI: 10.1038/nbt.3199. Crossref, Medline, Google Scholar
42. Bhatt S, Chalmers R. Targeted DNA transposition in vitro using a dCas9-transposase fusion protein. Nucleic Acids Res 2019; 47:8126-8135. DOI: 10.1093/nar/gkz552. Crossref, Medline, Google Scholar
43. Pickens L B, Tang Y, Chooi Y H. Metabolic engineering for the production of natural products. Annu Rev Chem Biomol Eng 2011; 2:211-236. DOI: 10.1146/annurev-chembioeng-061010-114209. Crossref, Medline, Google Scholar
44. Esvelt K M, Smidler A L, Catteruccia F, et al. Concerning RNA-guided gene drives for the alteration of wild populations. eLife 2014; 3. DOI: 10.7554/eLife.03401. Google Scholar
45. Ronda C, Chen S P, Cabral V, et al. Metagenomic engineering of the mammalian gut microbiome in situ. Nat Methods 2019; 16:167-170. DOI: 10.1038/s41592-018-0301-y. Crossref, Medline, Google Scholar
46. Rohland N, Reich D. Cost-effective, high-throughput DNA sequencing libraries for multiplexed target capture. Genome Res 2012; 22:939-946. DOI: 10.1101/gr.128124.111. Crossref, Medline, Google Scholar
47. Langmead B, Salzberg S L. Fast gapped-read alignment with Bowtie 2. Nat Methods 2012; 9:357-359. DOI: 10.1038/nmeth.1923. Crossref, Medline, Google Scholar
48. Sundaresan R, Parameshwaran H P, Yogesha S D, et al. RNA-independent DNA cleavage activities of Cas9 and Cas12a. Cell Rep 2017; 21:3728-3739. DOI: 10.1016/j.celrep.2017.11.100. Crossref, Medline, Google Scholar
49. Vigdal T J, Kaufman C D, Izsvak Z, et al. Common physical properties of DNA affecting target site selection of sleeping beauty and other Tc1/mariner transposable elements. J Mol Biol 2002; 323:441-452. DOI: 10.1016/s0022-2836(02)00991-9. Crossref, Medline, Google Scholar
50. Trubitsyna M, Morris E R, Finnegan D J, et al. Biochemical characterization and comparison of two closely related active mariner transposases. Biochemistry 2014; 53:682-689. DOI: 10.1021/bi401193w. Crossref, Medline, Google Scholar
51. Milo R, Jorgensen P, Moran U, et al. BioNumbers—the database of key numbers in molecular and cell biology. Nucleic Acids Res 2010; 38:D750-753. DOI: 10.1093/nar/gkp889. Crossref, Medline, Google Scholar
52. Lampe D J. Bacterial genetic methods to explore the biology of mariner transposons. Genetica 2010; 138:499-508. DOI: 10.1007/s10709-009-9401-z. Crossref, Medline, Google Scholar
53. Warming S, Costantino N, Court D L, et al. Simple and highly efficient BAC recombineering using galK selection. Nucleic Acids Res 2005; 33:e36. DOI: 10.1093/nar/gni035. Crossref, Medline, Google Scholar
54. Li X T, Thomason L C, Sawitzke J A, et al. Positive and negative selection using the tetA-sacB cassette: recombineering and P1 transduction in Escherichia coli. Nucleic Acids Res 2013; 41:e204. DOI: 10.1093/nar/gkt1075. Crossref, Medline, Google Scholar
55. DeVito J A. Recombineering with tolC as a selectable/counter-selectable marker: remodeling the rRNA operons of Escherichia coli. Nucleic Acids Res 2008; 36:e4. DOI: 10.1093/nar/gkm1084. Crossref, Medline, Google Scholar
56. Liu D, Chalmers R. Hyperactive mariner transposons are created by mutations that disrupt allosterism and increase the rate of transposon end synapsis. Nucleic Acids Res 2014; 42:2637-2645. DOI: 10.1093/nar/gkt1218. Crossref, Medline, Google Scholar

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A fusion protein comprising a transposase fused to a Cas protein, wherein the transposase is Himar1 or Tn5.

2. (canceled)

3. The fusion protein of claim 1, wherein the transposase comprises a polypeptide sequence comprising at least 80%, 85%, 90%, 91%, 92%, 93%, 94%, 95%, 96%, 97%, 98% or 99% sequence identity to the amino acid sequence of SEQ ID NO: 1, or 4, or active fragments thereof.

4. (canceled)

5. The fusion protein of claim 1, wherein the Cas protein is Cas9.

6. The fusion protein of claim 5, wherein the Cas9 protein is catalytically dead.

7-9. (canceled)

10. The fusion protein of claim 1, wherein the fusion protein comprises a polypeptide sequence comprising at least 80%, 85%, 90%, 91%, 92%, 93%, 94%, 95%, 96%, 97%, 98% or 99% sequence identity to the amino acid sequence of SEQ ID NO:3.

11. The fusion protein of claim 10, wherein the fusion protein comprises one or more mutations selected from the group consisting of Y12A, Y12S, F31A, W119A, V120A, P121A, R122A, E123A, and L124A.

12-13. (canceled)

14. The fusion protein of claim 1, wherein the fusion protein comprises a polypeptide sequence comprising at least 80%, 85%, 90%, 91%, 92%, 93%, 94%, 95%, 96%, 97%, 98% or 99% sequence identity to the amino acid sequence of SEQ ID NO:5.

15. The fusion protein of claim 14, wherein the fusion protein comprises one or more mutations selected from the group consisting of M470_I476del, A471_I476del, and S458A.

16. (canceled)

17. A system comprising a fusion protein according to claim 1 and at least one gRNA sequence complementary to a segment of DNA sequence, wherein the segment is adjacent to a target site of a target nucleic acid.

18-20. (canceled)

21. The system of claim 17, further comprising at least one mini-transposon.

22. The system of claim 21, wherein the mini-transposon comprises a payload sequence comprising a 5′ and 3′ end, a first transposon end sequence that is fused to the 5′ end of a payload sequence and a second transposon end sequence that is fused at the 3′ end of the payload sequence.

23. The system of claim 21, wherein the transposon end sequence comprises an inverted repeat of a Himar1 transposon or Tn5 transposon.

24. The system of claim 22, wherein the transposon end sequence comprises a sequence having at least 80%, 85%, 90%, 91%, 92%, 93%, 94%, 95%, 96%, 97%, 98% or 99% sequence identity with SEQ ID NO:9, or reverse complement thereof, or SEQ ID NO:12, or a reverse complement thereof.

25. The system of claim 17, wherein the at least one gRNA sequence comprises a first gRNA sequence that is complementary to a first DNA segment of the target nucleic acid and a second gRNA sequence that is complementary to a second DNA segment of the target nucleic acid.

26. A method of inserting a transposon into a target site of a target nucleic acid to disrupt expression of the target nucleic acid, the method comprising providing to the target nucleic acid (i) a fusion protein of claim 1, and (ii) at least one gRNA sequence complementary to a segment of a target nucleic acid, wherein the segment is adjacent to the target site to direct transposon insertion, and, optionally, (iii) at least one mini-transposon.

27. The method of claim 26, wherein elements (i), (ii), and (iii) are packaged into a single vector.

28-30. (canceled)

31. The method of claim 26, wherein the target nucleic acid is a DNA sequence in a cell.

32. The method of claim 26, wherein the at least one gRNA sequence comprises a first gRNA sequence that is complementary to a first DNA segment of the target nucleic acid and a second gRNA sequence that is complementary to a second DNA segment of the target nucleic acid.

33. The method of claim 26, wherein any of elements (i), (ii) and/or (iii) are synthesized in vitro and then delivered to a cell or cell-free system.

34-66. (canceled)

67. The method of claim 26, wherein the mini-transposon comprises a payload sequence comprising a 5′ and 3′ end, a first transposon end sequence that is fused to the 5′ end of a payload sequence and a second transposon end sequence that is fused at the 3′ end of the payload sequence.


SEQUENCE LISTING (57 sequences)

SEQ ID NO: 1 - protein, 348 aa, Haematobia irritans
  misc_feature: Himar1 WT
MEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQQRVDDSERCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVE

SEQ ID NO: 2 - protein, 348 aa, Haematobia irritans
  misc_feature: Himar1C9
MEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQRRVDDSKRCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVE

SEQ ID NO: 3 - protein, 1732 aa, Artificial Sequence
  Synthetic Himar1C9-dCas9 fusion protein
MEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQRRVDDSKRCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVESGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 476 aa, Artificial Sequence
  Synthetic Hyperactive Tn5 transposase
MITSALHRAADWAKSVFSSAALGDPRRTARLVNVAAQLAKYSGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 5 - protein, 1860 aa, Artificial Sequence
  Synthetic Tn5-dCas9 fusion protein with XTEN linker
MITSALHRAADWAKSVFSSAALGDPRRTARLVNVAAQLAKYSGKSITISSEGSKAAQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKISGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1368 aa, Artificial Sequence
  Synthetic dCas9 (D10A, H840A)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1775 aa, Artificial Sequence
  Synthetic Himar1C9-dCas9 fusion protein with N-terminus 3xFLAG and SV40 mammalian NLS
MDYKDHDGDYKDHDIDYKDDDDKMAPKKKRKVGIHRGVPGGSGSMEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQRRVDDSKRCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVESGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1745 aa, Artificial Sequence
  Synthetic Himar1C9-dCas9 fusion protein with C-terminal E. coli SsrA degradation tag
MEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQRRVDDSKRCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVESGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDRPAANDENYALAA

SEQ ID NO: 9 - DNA, 27 bp, Artificial Sequence
  Synthetic Himar1 Transposon inverted repeat
acaggttggatgataagtccccggtct

SEQ ID NO: 10 - DNA, 1375 bp, Artificial Sequence
  Synthetic Himar1 mini-transposon containing chloramphenicol resistance cassette as payload (from plasmid pHimar6)
acaggttggatgataagtccccggtcttcgtatgccgtcttctgcttggcgcgccctcgagcaattgccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttatactctagagtagtagattattttaggaatttagatgttttgtatgaaatagatgcttcgtatggaattaatgaaatttttagtcaggtaaaaaaggtaataggagaatattatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaaacaataggccacatgcaactgtctagaatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacctgcagggcgcgccaagcagaagacggcatacgaagaccggggacttatcatccaacctgt

SEQ ID NO: 11 - DNA, 5199 bp, Artificial Sequence
  Synthetic DNA coding sequence for Himar1C9- dCas9 fusion protein with XTEN linker
atggaaaaaaaggaatttcgtgttttgataaaatactgttttctgaagggaaaaaatacagtggaagcaaaaacttggcttgataatgagtttccggactctgccccagggaaatcaacaataattgattggtatgcaaaattcaagcgtggtgaaatgagcacggaggacggtgaacgcagtggacgcccgaaagaggtggttaccgacgaaaacatcaaaaaaatccacaaaatgattttgaatgaccgtaaaatgaagttgatcgagatagcagaggccttaaagatatcaaaggaacgtgttggtcatatcattcatcaatatttggatatgcggaagctctgtgcgaaatgggtgccgcgcgagctcacatttgaccaaaaacaacgacgtgttgatgattctaagcggtgtttgcagctgttaactcgtaatacacccgagtttttccgtcgatatgtgacaatggatgaaacatggctccatcactacactcctgagtccaatcgacagtcggctgagtggacagcgaccggtgaaccgtctccgaagcgtggaaagactcaaaagtccgctggcaaagtaatggcctctgttttttgggatgcgcatggaataatttttatcgattatcttgagaagggaaaaaccatcaacagtgactattatatggcgttattggagcgtttgaaggtcgaaatcgcggcaaaacggccccacatgaagaagaaaaaagtgttgttccaccaagacaacgcaccgtgccacaagtcattgagaacgatggcaaaaattcatgaattgggcttcgaattgcttccccacccgccgtattctccagatctggcccccagcgactttttcttgttctcagacctcaaaaggatgctcgcagggaaaaaatttggctgcaatgaagaggtgatcgccgaaactgaggcctattttgaggcaaaaccgaaggagtactaccaaaatggtatcaaaaaattggaaggtcgttataatcgttgtatcgctcttgaagggaactatgttgaaagcggttccgaaactcccggtacatcagaaagcgcgacccccgaaagcatggataaaaagtattctattggtttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 12 - DNA, 19 bp, Artificial Sequence
  Synthetic Tn5 transposon inverted repeat
ctgtctcttatacacatct

SEQ ID NO: 13 - DNA, 1041 bp, Artificial Sequence
  Synthetic Tn5 mini-transposon containing chloramphenicol resistance cassette as payload
ctgtctcttatacacatctcaaccatcatcgatgaattttctcgggtgttctcgcatattggctcgaattcctgcagcccctctagagtagtagattattttaggaatttagatgttttgtatgaaatagatgcttcgtatggaattaatgaaatttttagtcaggtaaaaaaggtaataggagaatattatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaaacaataggccacatgcaactgtctagaatgcgagagtagggaactgccaggcatcaaataaaacgaaaggctcagtcgaaagactgggcctttcgttttattgaacggtagcatcttgacgacgcagcttgccaacgactacgcactagccaacaagagcttcagggttgagatgtgtataagagacag

SEQ ID NO: 14 - DNA, 120 bp, Artificial Sequence
  Synthetic Fig. 8 sequence
catgacttcttcaagtccgccatgccggaaggctatgtgcaggacgcacgatttcctttaaggatgacggcacgtacaaaacgcgtgcggaagtgaaatttgaaggcgataccctggtaa

SEQ ID NO: 15 - protein, 30 aa, Artificial Sequence
  Synthetic peptide linker
  misc_feature (4)..(30): Any “Gly Gly Ser” may or may not be present
GGSGGSGGSGGSGGSGGSGGSGGSGGSGGS

SEQ ID NO: 16 - protein, 18 aa, Artificial Sequence
  Synthetic peptide linker
GGSGGSGGSGGSGGSGGS

SEQ ID NO: 17 - DNA, 20 bp, Artificial Sequence
  Synthetic gRNA_1
gtcgttaccagagtcggcca

SEQ ID NO: 18 - DNA, 20 bp, Artificial Sequence
  Synthetic gRNA_2
tcagtgctttgctcgttatc

SEQ ID NO: 19 - DNA, 20 bp, Artificial Sequence
  Synthetic gRNA_3
cgttcctgcacatagccttc

SEQ ID NO: 20 - DNA, 20 bp, Artificial Sequence
  Synthetic gRNA_4
cggcacgtacaaaacgcgtg

SEQ ID NO: 21 - DNA, 20 bp, Artificial Sequence
  Synthetic gRNA_5
gtcggcggggtgcttcacgt

SEQ ID NO: 22 - DNA, 20 bp, Artificial Sequence
  Synthetic gRNA_7
accagagtcggccaaggtac

SEQ ID NO: 23 - DNA, 20 bp, Artificial Sequence
  Synthetic gRNA_8
ctgcacatagccttccggca

SEQ ID NO: 24 - DNA, 20 bp, Artificial Sequence
  Synthetic gRNA_9
caatgcctttcagctcaatg

SEQ ID NO: 25 - DNA, 20 bp, Artificial Sequence
  Synthetic gRNA_10
cagctcaatgcggtttacca

SEQ ID NO: 26 - DNA, 20 bp, Artificial Sequence
  Synthetic gRNA_11
gtaaaccgcattgagctgaa

SEQ ID NO: 27 - DNA, 20 bp, Artificial Sequence
  Synthetic gRNA_12
caatatcctgggccataagc

SEQ ID NO: 28 - DNA, 20 bp, Artificial Sequence
  Synthetic gRNA_13
agaacaggaccatcaccgat

SEQ ID NO: 29 - DNA, 20 bp, Artificial Sequence
  Synthetic gRNA_14
gtgctcagatagtgattgtc

SEQ ID NO: 30 - DNA, 20 bp, Artificial Sequence
  Synthetic gRNA_15
gaactggatggtgatgtcaa

SEQ ID NO: 31 - DNA, 20 bp, Artificial Sequence
  Synthetic gRNA_16
ccttccccgagggcttcaag

SEQ ID NO: 32 - DNA, 20 bp, Artificial Sequence
  Synthetic gRNA_18
acgcgatcacatggttctgc

SEQ ID NO: 33 - DNA, 25 bp, Artificial Sequence
  Synthetic p433
cgcttacaatttccattcgccattc

SEQ ID NO: 34 - DNA, 22 bp, Artificial Sequence
  Synthetic p415
ccctgcaaagcccctctttacg

SEQ ID NO: 35 - DNA, 20 bp, Artificial Sequence
  Synthetic p828
ctgcgcaacccaagtgctac

SEQ ID NO: 36 - DNA, 23 bp, Artificial Sequence
  Synthetic p829
cagtccagagaaatcggcattca

SEQ ID NO: 37 - DNA, 22 bp, Artificial Sequence
  Synthetic p923
  modified_base (1)..(1): 5′ biotin
gccataaactgccaggcatcaa

SEQ ID NO: 38 - DNA, 19 bp, Artificial Sequence
  Synthetic p922
ccttcttgcgcatctcacg

SEQ ID NO: 39 - DNA, 34 bp, Artificial Sequence
  Synthetic Adapter_T
  modified_base (1)..(1): 5′ Phosphate
agatcggaagagcacacgtctgaactccagtcac

SEQ ID NO: 40 - DNA, 30 bp, Artificial Sequence
  Synthetic Adapter_B
  misc_feature (29)..(30): n is a, c, g, or t
gtctcgtgggctcgggctcttccgatctnn

SEQ ID NO: 41 - DNA, 68 bp, Artificial Sequence
  Synthetic p790
aatgatacggcgaccaccgagatctacactagatcgccgccagaccggggacttatcatccaacctgt

SEQ ID NO: 42 - DNA, 68 bp, Artificial Sequence
  Synthetic p791
aatgatacggcgaccaccgagatctacacctctctatcgccagaccggggacttatcatccaacctgt

SEQ ID NO: 43 - DNA, 68 bp, Artificial Sequence
  Synthetic p792
aatgatacggcgaccaccgagatctacactatcctctcgccagaccggggacttatcatccaacctgt

SEQ ID NO: 44 - DNA, 68 bp, Artificial Sequence
  Synthetic p793
aatgatacggcgaccaccgagatctacacagagtagacgccagaccggggacttatcatccaacctgt

SEQ ID NO: 45 - DNA, 68 bp, Artificial Sequence
  Synthetic p794
aatgatacggcgaccaccgagatctacacgtaaggagcgccagaccggggacttatcatccaacctgt

SEQ ID NO: 46 - DNA, 68 bp, Artificial Sequence
  Synthetic p795
aatgatacggcgaccaccgagatctacacactgcatacgccagaccggggacttatcatccaacctgt

SEQ ID NO: 47 - DNA, 31 bp, Artificial Sequence
  Synthetic p712
cgccagaccggggacttatcatccaacctgt

SEQ ID NO: 48 - DNA, 24 bp, Artificial Sequence
  Synthetic p713
cggaagagcccgagcccacgagac

SEQ ID NO: 49 - DNA, 22 bp, Artificial Sequence
  Synthetic p898
tttgagtgagctgataccgctc

SEQ ID NO: 50 - DNA, 22 bp, Artificial Sequence
  Synthetic p899
gagcggtatcagctcactcaaa

SEQ ID NO: 51 - DNA, 24 bp, Artificial Sequence
  Synthetic p900
tcccttaacgtgagttttcgttcc

SEQ ID NO: 52 - protein, 16 aa, Artificial Sequence
  Synthetic flexible protein linker XTEN35
SGSETPGTSESATPES

SEQ ID NO: 53 - DNA, 49 bp, Artificial Sequence
  Synthetic
gtcgttaccagagtcggccaguuuuagagcuagaaauagaaguuaaaau

SEQ ID NO: 54 - DNA, 49 bp, Artificial Sequence
  Synthetic
tcagtgctttgctcgttatcguuuuagagcuagaaauagaaguuaaaau

SEQ ID NO: 55 - DNA, 64 bp, Artificial Sequence
  Synthetic
taccttggccgactctggtaacgacgctgacttatggtgttcagtgctttgctcgttatccgga

SEQ ID NO: 56 - DNA, 18 bp, Artificial Sequence
  Synthetic
gcgaattttaaaattcgc

SEQ ID NO: 57 - DNA, 120 bp, Artificial Sequence
  Synthetic
gaagtgaaatttgaaggcgataccctggtaaaccgcattgagctgaaaggcattgactttaaagaagacggcaatatcctgggccataagctggaatacaattttaacagccacaatgtt